(19) United States
(12) Patent Application Publication
(10) Pub. No.: US 2012/0042398 A1
(43) Pub. Date: Feb. 16, 2012

(54) COMPOSITIONS FOR LABELING AND IDENTIFYING AUTOPHAGOSOMES AND METHODS FOR MAKING AND USING THEM

(75) Inventors: Roberta A. Gottlieb, Solana Beach, CA (US); Thomas E. Cole, San Diego, CA (US); Cynthia N. Perry-Garza, San Diego, CA (US); Raquel Sousa Carreira, San Diego, CA (US); Bryan J. Bartlett, San Diego, CA (US); Kim Finley, Poway, CA (US)

(73) Assignee: SAN DIEGO STATE UNIVERSITY (SDSU) FOUNDATION, San Diego, CA (US)

(21) Appl. No.: 13/123,704

(22) PCT Filed: Oct. 13, 2009
(86) PCT No.: PCT/US09/60563

§ 371 Date: Oct. 31, 2011

Related U.S. Application Data
(60) Provisional application No. 61/235,239, filed on Aug. 19, 2009.
(60) Provisional application No. 61/104,982, filed on Oct. 13, 2008.

(51) Int. Cl. A61K 49/00; C07F 5/02; A61K 9/127; A61K 51/00; A61K 31/69; C12Q 1/02; C40B 30/06; A61B 5/055; C07D 311/80; A61K 31/352

(52) U.S. Cl. 800/3; 549/391; 564/11; 424/450; 514/455; 514/64; 435/29; 506/10; 424/9.6; 424/9.3; 424/1.11

(57) ABSTRACT

The invention provides methods and compositions for detecting and measuring the amount of autophagosomes in cells or tissues, including biopsy samples, in vitro, in situ and/or in vivo. By detecting and measuring the amount of autophagosomes in cells or tissues, the methods and compositions of the invention also measure the amount of autophagic activity in a cell or a tissue. In one aspect, the invention can be adapted to a plate-reader format for high-throughput screening of drugs that modulate autophagy, i.e., high-throughput detection of autophagic (autophagosome) activity in cells or tissues. In alternative embodiments, the compositions of the invention can localize into autophagosomes (AV), and these compositions can comprise any detectable moiety or group, e.g., a cadaverine, a radioactive, fluorescent-, bioluminescent and/or paramagnetic-conjugated reagent.

### TECHNICAL FIELD

This invention relates to medicine, cellular biology and biochemistry. The invention provides methods and compositions for detecting and measuring the amount of autophagosomes in cells or tissues, including biopsy samples, in vitro, in situ and/or in vivo. By detecting and measuring the amount of autophagosomes in cells or tissues, the methods and compositions of the invention also measure the amount of autophagic activity in a cell or a tissue.

In one aspect, the invention can be adapted to a plate-reader format for high-throughput screening of drugs that modulate autophagy, i.e., high-throughput detection of autophagic (autophagosome) activity in cells or tissues. In alternative embodiments, the compositions used to practice this invention can localize into autophagosomes (AV) or subpopulations of AV, and these compositions can comprise any detectable or “reporter” group or domain, e.g., cadaverine derivative(s), fluorescent-, bioluminescent, radioactive- and/or paramagnetic-conjugated reagents.

In alternative embodiments, the compositions used to practice this invention and the methods of this invention are used to assess (e.g., to evaluate, diagnose, measure) autophagy in an individual's (e.g., patient's) tissues in several settings, including detecting whether or not a particular drug or intervention is effectively inducing or inhibiting autophagy. Because regulating autophagy is therapeutically important in the setting of myocardial ischemia/reperfusion injury, neurodegenerative diseases (such as Alzheimer's disease, Lewy Body Disease, Parkinson's Disease, Huntington's Disease, Multi-infarct dementia, senile dementia or Frontotemporal Dementia), diabetes, atherosclerosis, cardiac hypertrophy, heart failure, glycogen storage disease type II (also called Pompe disease or acid maltase deficiency), and many other conditions, in alternative embodiments the compositions and the methods of this invention are used to assess (evaluate) the effectiveness of a treatment or prophylactive drug for myocardial ischemia/reperfusion injury, neurodegenerative diseases (such as Alzheimer's disease, Lewy Body Disease, Parkinson's Disease, Huntington's Disease, Multi-infarct dementia, senile dementia or Frontotemporal Dementia), diabetes, atherosclerosis, cardiac hypertrophy, heart failure, and/or glycogen storage disease type II and/or many other conditions.

### BACKGROUND

To date, there have been no reliable assays of autophagy, e.g., that are suitable for plate-reader format, and even more importantly, current methods for assessing autophagy in organs or tissues from live mammals are extremely limited. Currently no methods exist for assessing autophagy in situ in the living mammal.

The current industry standard is to transfect cells with green fluorescent protein-tagged autophagic marker protein light chain 3 (GFP-LC3) (see e.g., Gonzalez-Polo R-A, et al. (2005) J. Cell Sci. 118:3091-3102), which is a fluorescent fusion protein that is incorporated into autophagosomes (also called autophagic vesicles, or AV), and to then use confocal microscopy to score the number of autophagosomes (LC3-GFP dots) per cell. Although this can be done using robotics and automated microscopy, it is cumbersome and requires the use of cell lines that are transiently or stably transfected with LC3-GFP. Since the transfection procedure and overexpression of LC3-GFP can influence the basal level of autophagy, some degree of artifact is introduced into the assay. Moreover, not all cells or cell lines can be transfected efficiently, and the assay is rather cumbersome.

Current methods to measure autophagy in vivo obtain biopsy material, fix and embed the tissue, section it, and perform immunohistochemistry to detect autophagosomes using antibody to LC3 followed by visual inspection and manual scoring of the number of labeled structures per unit area in the section. This is not an accurate quantitative procedure, as only a few fields of the tissue section may be scored, often representing less than 10% of the entire biopsy sample. The only way to normalize for cell number or to account for area occupied by intracellular structures is to manually or subjectively make an assessment of autophagosome number. Electron microscopy is often performed to confirm the finding, as autophagosomes are double-membrane structures, but confines the assessment to an even smaller section of tissue, often only a few cells. Both of these procedures are costly and time-consuming, requiring several days for tissue processing, considerable expertise, and extensive time performing the microscopic imaging and scoring. Other methods rely on biochemical measurement of autophagy-related proteins such as LC3-II.

There are currently no methods to assess autophagy in a given tissue in the living organism, except to use intravital microscopy in transgenic mice or other model organisms that are expressing fluorescent LC3 (GFP-LC3 or mCherry-LC3), where the organ or tissue of interest is accessible to the microscope.

### SUMMARY

The invention provides methods and compositions for detecting and measuring the amount of autophagosomes in cells or tissues, including biopsy samples, in vitro, in situ and/or in vivo. By detecting and measuring the amount of autophagosomes in cells or tissues, the methods and compositions of the invention also measure the amount of autophagic activity in a cell or a tissue, including measuring autophagic activity in a cell or a tissue biopsy sample, in vitro, in situ and/or in vivo.

In alternative embodiments, the invention provides chimeric molecules comprising at least two domains (or moieties or groups) comprising:

(a) a first domain or moiety (or group) comprising: a primary amine; a bifurcated di- or triamine, a tertiary amine; a polyamine; an N,N-dimethyl or diethyl amine; an aliphatic amine; a heteroaromatic amine, an ethylenediamine, a 1,3-diaminopropane, a 1,4-diaminobutane, a 1,6 diaminohexane, a 2,2′ (ethylenedioxy)diethylamine, a triethylene glycol diamine, an N,N-dimethylaniline, a guanidine, a spermine or a spermidine (linear), or a structure selected from the group consisting of

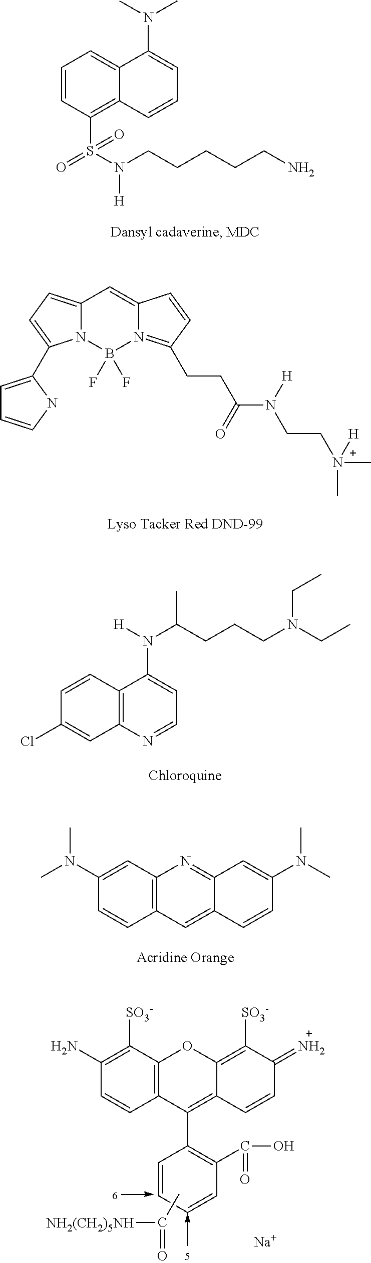

AlexaFluor 488 ™ cadaverine, or other fluor-conjugated cadaverine molecules (see list)

Alexa Fluor® 647 azide, triethylammonium salt

Alexa Fluor® 350 cadaverine

Alexa Fluor® 405 cadaverine, trisodium salt

Alexa Fluor® 488 cadaverine, sodium salt

Alexa Fluor® 555 cadaverine, disodium salt

Alexa Fluor® 568 cadaverine, diammonium salt

Alexa Fluor® 594 cadaverine

Alexa Fluor® 647 cadaverine, disodium salt

fluo-4 cadaverine, pentapotassium salt

Oregon Green® 488 cadaverine *5-isomer*

Texas Red® cadaverine (Texas Red® C₅)

5-(((4-(4,4-difluoro-5-(2-thienyl)-4-bora-3a,4a-diaza-s-indacene-3-yl)phenoxy)acetyl)amino)pentylamine, hydrochloride (BODIPY® TR cadaverine)

or equivalents thereof, or derivatives thereof, or any combination thereof;

(b) a second domain or moiety (or group) comprising a detectable or “reporter” composition or moiety; and

(c) a spacer, linker or direct coupling agent covalently or non-covalently (e.g., electrostatically) joining the first domain or moiety to the second domain or moiety, wherein the chimeric molecule is capable of localizing to (e.g., detecting, or binding to) an autophagosome (or autophagic vesicle, or AV) to detect and/or measure the amount of autophagic activity in a cell extract, a cell, a tissue, an organ or an organism, wherein optionally the chimeric molecule is capable of localizing to (detecting, or binding to) one or more AV sub-populations to detect and/or measure the amount of the one or more AV subpopulation(s), wherein optionally the one or more AV subpopulation(s) comprises an autophagosome AV subpopulation, an autolysosome AV subpopulation or a lysosomal vesicle AV subpopulation.

In alternative embodiments, compounds of the invention comprise four primary components: a reporter group, a linker bond, a linker and a reactive head group. These components cooperatively influence the overall properties of these compounds of the invention (which can act acts dyes and labels) in biological systems in terms of their selectivity, specificity and stability; and the choice of any particular reporter group, linker bond, linker and/or reactive head group can be selected based on the particular indication or desired use (e.g., high-throughput screening of drugs) and/or which AV subgroup is desired to be targeted, labeled and/or measured. For example, in alternative aspects, compounds of the invention selectively label autophagic vesicles (AVs), or selectively label a subset of AVs, wherein the AV subpopulation can comprise an autophagosome AV subpopulation, an autolysosome AV subpopulation and/or a lysosomal vesicle AV subpopulation.

In alternative embodiments of the chimeric molecules, the detectable or “reporter” composition or moiety comprises a radioactive, a radio-opaque, a fluorescent, bioluminescent and/or paramagnetic composition or moiety, or heavy metals for TEM. The detectable or “reporter” composition or moiety comprises a dansyl, a monodansyl, a fluorescein, a fluorescein isothiocyanate (FITC), a boron-dipyrromethene (BODIPY, or 4,4-difluoro-4-bora-3a,4a-diaza-s-indacene), a BODIPY-TR™, an ALEXA FLUOR™ dye (Molecular Probes, Life Sciences, Carlsbad, Calif.), an ALEXAFLUOR488™, a DYLIGHT™ fluor (Thermo Fisher Scientific, Waltham, Mass.), a DYLIGHT 488™ fluor, an ATTO™ dye (ATTO-TEC, GmbH, Siegen, Germany), a HILYTE dye (AnaSpec Inc., San Jose, Calif.), a positron-emitting agent, a Fluorine-18, a Carbon-11, a quantum dot nanoparticle, a gadolinium or a ferritin and nanoparticles of heavy metals.

In alternative embodiments of the chimeric molecules, the spacer, linker or direct coupling agent comprises a peptide or a synthetic molecule, or the spacer, linker or direct coupling agent comprises a thiourea, a sulfonamide or an amide. The peptide or synthetic molecule can comprise a polyglycine; a polyethylene glycol; a peptide comprising glycine, serine, threonine and/or alanine; a carbodiimide; a sulfhydryl-reactive composition; a glutaraldehyde or a glutardialdehyde (pentanedial); a hetero-bifunctional photoreactive phenylazide; a N-hydroxy-succinimidyl-comprising composition; or a combination thereof; or a structure selected from the group consisting of:

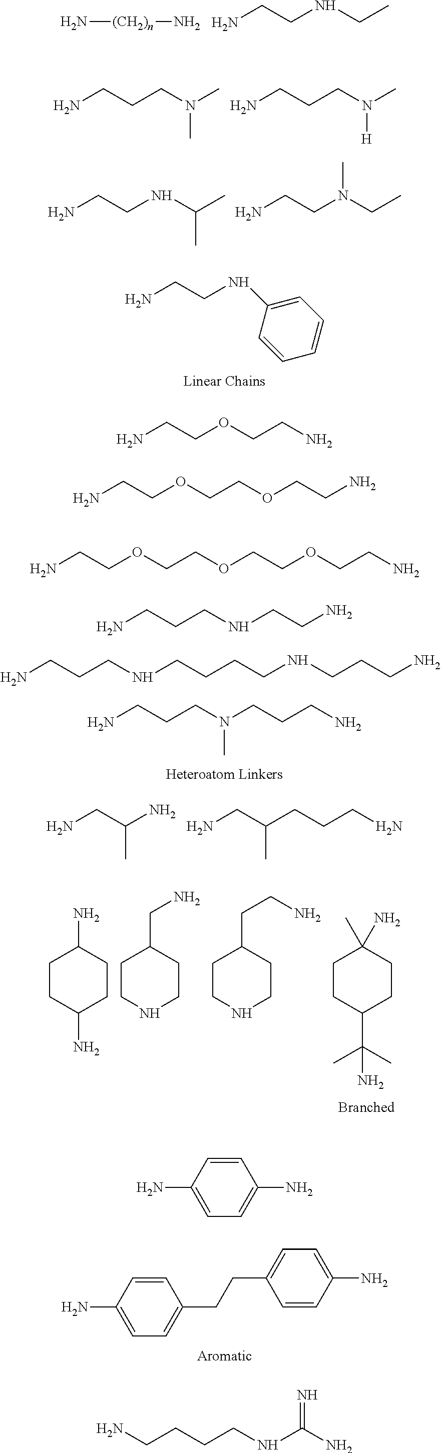

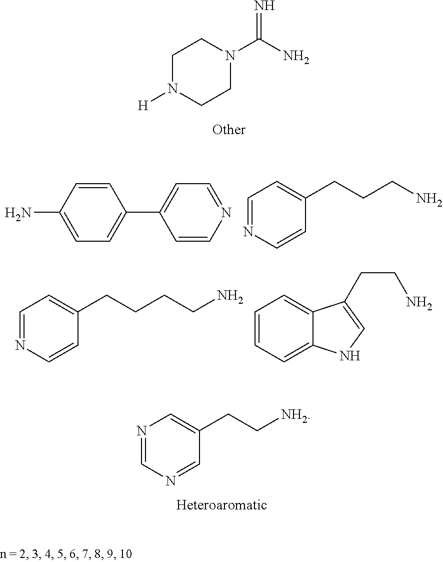

In alternative embodiments of the chimeric molecules, the carbodiimide can comprise dicyclohexylcarbodiimide (DCC), diisopropylcarbodiimide (DIC) or N′-(3-dimethylaminopropyl)-N-ethylcarbodiimide hydrochloride (EDC); or the sulfhydryl-reactive composition comprises a maleimide, a pydridyldisulfide, an alpha-haloacetyl, a vinylsulfone or a sulfatoalkylsulfone; the hetero-bifunctional photoreactive phenylazide comprises a sulfosuccinimidyl-2-(p-azido salicylamido)ethyl-1,3′-dithiopropionate; the N-hydroxy-succinimidyl-comprising composition comprises N-Succinimidyl-S-acetylthioacetate (SATA), a N-Succinimidyl 3-(2-pyridyldithio)-propionate) (SPDP), a Succinimidyl 6-(3-[2-pyridyldithio]-propionamido)hexanoate) (LC-SPDP), or a (N-Succinimidyl[4-iodoacetyl]aminobenzoate) (SIAB), or any combination of equivalent thereof.

In alternative embodiments, the invention provides liposomes comprising: (a) one or more chimeric molecules of the invention; or (b) the liposome of (a), wherein optionally the liposome is formulated with a pharmaceutically acceptable excipient or a buffer.

In alternative embodiments, the invention provides pharmaceutical compositions or formulations comprising: (a) one or more chimeric molecules of the invention, or at least one liposome of the invention; or (b) the pharmaceutical composition or formulation of (a), wherein the pharmaceutical composition or formulation is formulated with a pharmaceutically acceptable excipient.

In alternative embodiments, the invention provides inhalants or spray formulations comprising: one or more chimeric molecules of the invention, at least one liposome of the invention, or at least one pharmaceutical composition of the invention; and, a pharmaceutically acceptable excipient.

In alternative embodiments, the invention provides parenteral formulations comprising: one or more chimeric molecules of the invention, at least one liposome of the invention, or at least one pharmaceutical composition of the invention; and, a pharmaceutically acceptable excipient; or (b) the parenteral formulation of (a) formulated for intravenous, subcutaneous, intrathecal or intramuscular administration.

In alternative embodiments, the invention provides enteral formulations comprising: (a) one or more chimeric molecules of the invention, at least one liposome of the invention, at least one pharmaceutical composition of the invention, or the inhalant or spray of the invention; and, a pharmaceutically acceptable excipient; or (b) the enteral formulation of (a) formulated for oral, rectal or sublingual administration.

In alternative embodiments, the invention provides methods for detecting or measuring the amount of autophagic activity in a cell extract, a cell, a tissue, an organ or an organism, or detecting or binding or measuring the amount of to an autophagosome (or autophagic vesicle, or AV), in a cell extract, a cell, a tissue, an organ or an organism, comprising:

(a) providing one or more chimeric molecules of the invention, at least one liposome of the invention, at least one pharmaceutical composition of the invention, an inhalant or spray of the invention, a parenteral formulation of the invention, or the enteral formulation of the invention;

(b) contacting the chimeric molecule, the liposome, the pharmaceutical composition or formulation, the inhalant or spray, the parenteral formulation or the enteral formulation with the cell extract, cell, tissue, organ or organism; and

(c) detecting the presence and amount of the detectable composition or moiety; and optionally further comprising detecting the location of the chimeric molecules in the cell extract, cell, tissue, organ or organism,

wherein optionally the chimeric molecule is capable of localizing to (e.g., including detecting, or binding to) an AV sub-population to detect and/or measure the amount of the AV subpopulation,

wherein optionally the AV subpopulation comprises an autophagosome AV subpopulation, an autolysosome AV subpopulation or a lysosomal vesicle AV subpopulation.

In alternative embodiments of the methods of the invention, the detecting step (c) comprises use of a fiberoptic catheter or needle comprising a detecting device for detecting and measuring the amount of the detectable composition or moiety in a cell, tissue, organ or organism, and/or comprises use of a fluorimeter or luminometer attached to a fiberoptic probe.

In alternative embodiments, the method can comprise (a) use of a paramagnetic agent injected into a cell, tissue, organ or organism, and the amount of the detectable composition or moiety incorporated into the cell, tissue, organ or organism is a indicator of the extent of autophagy in that site; (b) the method of (a), wherein the amount of the detectable composition or moiety is assessed (measured) using nuclear magnetic resonance (NMR or MRI) imaging; or (c) the method of (a) or (b), wherein the detectable composition or moiety comprises a gadolinium or a ferritin.

In alternative embodiments of the methods of the invention, the method comprises (a) the detectable composition or moiety comprises a positron-emitting agent injected into a cell, tissue, organ or organism, and the amount of the detectable composition or moiety incorporated into the cell, tissue, organ or organism is a indicator of the extent of autophagy in that site; (b) the method of (a), wherein the amount of the detectable composition or moiety is assessed (measured) using a positron emission tomography (PET) imaging; or (c) the method of (a) or (b), wherein the detectable composition or moiety comprises a Fluorine-18 or a Carbon-11 incorporated into the moiety.

In alternative embodiments of the methods of the invention, the cell, tissue, organ or organism sample is or comprises a biopsy sample and/or a cell extract.

The invention provides methods for screening, e.g., high-throughput screening, of drugs or reagents that modulate autophagy or the amount of autophagosomes (AV) or AV activity in a cell extract, cell, tissue, organ, organism or individual, comprising:

(a) providing one or more chimeric molecules of the invention;

(b) providing a test reagent or drug (a candidate drug or reagent to be screened for its ability to modulate autophagy);

(c) contacting one sample of (or derived from) a cell extract, cell, tissue, organ, organism or individual with the chimeric molecule (control sample), and contacting a second sample (equivalent to the first sample for comparative purposes) with the test reagent or drug and the chimeric molecule (test sample); and

(d) detecting the amount of autophagy, or the amount of autophagosomes (AV) or AV activity, in the cell extract, cell, tissue, organ, organism or individual with and without the test reagent or drug,

wherein an increase or a decrease in the amount of autophagy as compared to control (without test reagent or drug) indicates that the test reagent or drug is a modulator of autophagy in a cell extract, cell, tissue, organ, organism or individual,

wherein an increase or a decrease in the amount of the detectable composition or moiety as compared to control (without the detectable composition or moiety) in a cell extract, cell, tissue, organ, organism or individual indicates that the test reagent or drug is a modulator of autophagy in the cell extract, cell, tissue, organ, organism or individual.

In alternative embodiments of the screening methods of the invention, the method comprises use of fluorescence microscopy or a fluorescence imaging to determine the amount of and/or the location of the detectable composition or moiety in the cell extract, cell, tissue, organ, organism or individual. The screening, e.g., high-throughput screening, method can comprise high-content imaging on a multi-well plate. The screening can be constructed and practiced on a multi-well plate. Transmission electron microscopy (TEM) can be used to determine the amount of and/or the location of the detectable composition or moiety in the cell extract, cell, tissue, organ, organism or individual.

In one aspect, the invention can be adapted to a plate-reader format for high-throughput screening of drugs that modulate autophagy, i.e., high-throughput detection of autophagic (autophagosome) levels and/or activity in cells or tissues. In alternative embodiments, the compositions of the invention, e.g., cadaverine derivatives, that can localize into or detect autophagosomes (AV) or AV subpopulations, and these compositions can comprise any detectable moiety or group, e.g., cadaverine derivative(s), or fluorescent-, bioluminescent, radioactive- and/or paramagnetic-conjugated cadaverine reagents.

The invention provides methods for assessing (evaluating) the efficacy of a therapeutic or prophylactic (test) drug or composition by assessing its ability to modulate autophagy or modulate the amount and/or activity of autophagosomes (AV) in a cell extract, cell, tissue or organism or individual, comprising:

(a) providing one or more chimeric molecules of the invention;

(b) providing a therapeutic or a prophylactic drug or composition;

(c) contacting one sample of a cell extract, cell, tissue, organ or organism or individual with the chimeric molecule (control sample), and contacting a second sample (equivalent to the first sample for comparative purposes) with the therapeutic or prophylactic drug (test) drug and the chimeric molecule (test sample); and

(d) detecting the amount (levels) and/or activity of AVs and/or autophagy in the cell extract, cell, tissue, organ or organism or individual with and without the test reagent or drug,

wherein an increase or a decrease in the amount and/or activity of AVs and/or autophagy as compared to control (without test reagent or drug) indicates that the test reagent or drug is a modulator of levels and/or activity of AVs and/or autophagy in a cell extract, cell, tissue, organ or organism or individual,

wherein an increase or a decrease in the amount of detectable composition or moiety as compared to control (without detectable composition or moiety) in a cell extract, cell, tissue, organ or individual indicates that the test reagent or drug is a modulator of levels and/or activity of AVs and/or autophagy in the cell extract, cell extract, cell, tissue or organ or individual.

In alternative embodiments of the methods of the invention, the method assesses (evaluates) the efficacy of a therapeutic or prophylactic (test) drug for treating, ameliorating or preventing myocardial ischemia/reperfusion injury, a neurodegenerative disease, diabetes, atherosclerosis, cardiac hypertrophy, heart failure, glycogen storage disease type II (also called Pompe disease or acid maltase deficiency) and related conditions. The neurodegenerative disease can be Alzheimer's disease, Lewy Body Disease, Parkinson's Disease, Huntington's Disease, Multi-infarct dementia, senile dementia or Frontotemporal Dementia. The neurodegenerative disease can be related to or is a sequelae of a trauma, or exposure to a toxin or a poison.

In alternative embodiments of the methods of the invention, fluorescence microscopy or a fluorescence imaging is used to determine the amount of and/or the location of the detectable composition or moiety in the cell extract, cell, tissue or organ. Transmission electron microscopy (TEM) can be used to determine the amount of and/or the location of the detectable composition or moiety in the cell extract, cell, tissue or organ.

The invention provides kits comprising (a) a composition of the invention (e.g., a chimeric molecule of the invention), at least one liposome of the invention, at least one pharmaceutical composition of the invention, an inhalant or spray of the invention, a parenteral formulation of the invention, and/or the enteral formulation of the invention; or (b) the kit of (a), further comprising instruction for practicing a method of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The following drawings are illustrative of embodiments of the invention and are not meant to limit the scope of the invention as encompassed by the claims.

FIG. 1 illustrates the relative fluorescence of HL-1 cells loaded with ALEXAFLUOR488™-cadaverine, normalized to cell number (ethidium bromide fluorescence), as described in detail in Example 1, below.

FIG. 2 illustrates dye incorporation into autophagosomes after heart perfusion: FIG. 2's two images at left show heart tissue loaded with dye and an inducer of autophagy, with the lower left image also having co-administration of an autophagy inhibitor; FIG. 2's right image is a graph illustrating quantitation of dye incorporation into autophagosomes, with the left column graphically quantitating the upper left image's fluorescence and the right column graphically quantitating the lower left image's (the Tat-Atg5(K130R)-inhibited) fluorescence, as described in detail in Example 1, below.

FIG. 3A illustrates an Autophagy Pathway; FIG. 3B illustrates Dual ubiquitin-like pathways; FIG. 3C illustrates an image showing GFP-LC3-positive puncta in cardiomyoctyes with up-regulated autophagy; and FIG. 3D illustrates three images showing GFP-Atg8a (the left image) and LYSOTRACKER™ (red) stain (the right image) (and a merged image, the middle image) overlapping vesicles at a developmental period when autophagy is under hormonal control and up regulated, as described in detail in Example 2, below.

FIG. 4 illustrates three images, including mCherry and MDC labeling of vesicles: illustrated in the middle image of FIG. 4, is an MDC labeling showing a significant level of co-localization with mCherry-LC3 positive puncta (arrows in the right image); as illustrated in the left image of FIG. 4, mCherry-LC3-II highlights a subset of structures not stained by MDC; the right image of FIG. 4 is a merge of the mCherry-LC3 and MDC images, as described in detail in Example 2, below.

FIG. 5 illustrates an exemplary synthesis of Fluorescein- (FIG. 5A) and Texas Red- (FIG. 5B) conjugated Dyes for use in practicing this invention, as described in detail in Example 2, below.

FIG. 6 illustrates images showing fly tissues with activated autophagy that were collected and individually stained for 10 min with one of seven dyes (each of the seven samples were stained with only one dye). BODIPY-cadaverine was included as a positive control. The C-3, C-4, C-5 and C-6 dyes did not mark intracellular vesicles in fresh tissue preparations. The BODIPY-dye shows a robust staining pattern, as do the exemplary C-1 (FITC-ET) and C-2 (FITC-TG) compounds of the invention, as described in detail in Example 2, below.

FIG. 7 illustrates images showing: FIG. 7A and FIG. 7B: Fly larvae were fasted and fat body tissues stained with LYSOTRACKER™ and: either the exemplary (FIG. 7A) FITC-ET or (FIG. 7B) FITC-TG; FIG. 7C illustrates tissue from larvae undergoing hormone-triggered autophagy which was collected and stained with LYSOTRACKER™ and FITC-TG—and showing that similar staining was detected; FIG. 7D and FIG. 7E illustrate images showing fat was collected from larvae expressing GFP-Atg8a, fixed (3% PFA) and stained with: either the exemplary (FIG. 7D) Texas Red-ET or the exemplary (FIG. 7E) Texas Red-TG, as described in detail in Example 2, below.

FIG. 8 illustrates images of stained tissues from: Fed (left column images), fasted (middle column images) and hormone-induced (“3^{rd} instar” right column images) autophagy profiles in wildtype (upper row images) and Atg1−/− mutant (lower row images) cells, as described in detail in Example 2, below.

FIG. 9 illustrates images of the labeling of mouse and human tissue culture cells with exemplary dyes of the invention, as indicated in the figure images: left image is cardiac HL-1 cells stained with FITC-TG label; the next image is neural HT-22 cells with Tx-red-TG label; next is neural HT-22 cells stained with Tx-red-ET label; the right image is neural MC-65 cells with Tx-red-TG label, as described in detail in Example 2, below.

FIG. 10 illustrates an exemplary chemical reaction strategy to generate exemplary fluorescent dyes and compounds of the invention, as described in detail in Example 2, below.

FIG. 11 illustrates exemplary (representative) examples of commercially available linker and diamine head groups that can be used in compounds of this invention, as described in detail in Example 2, below.

FIG. 12 illustrates exemplary compounds of this invention that preferentially label acidified organelles, including lysosomes and autophagosome, as described in detail in Example 2, below.

Like reference symbols in the various drawings indicate like elements.

### DETAILED DESCRIPTION

The invention provides methods and compositions for measuring the amount of autophagic activity in cells or tissues, including biopsy samples, in vitro and/or in vivo. In one aspect, the invention can be adapted to a plate-reader format for high-throughput screening of drugs that modulate autophagy, i.e., high-throughput detection of autophagic (autophagosome) activity in cells or tissues. In alternative embodiments, the compositions used to practice this invention localize into autophagosomes (AV) and/or AV subpopulations, and these compositions can comprise any group or moiety, e.g., cadaverine derivatives, or fluorescent-, bioluminescent, radioactive- and/or paramagnetic-conjugated reagents.

In alternative embodiments, the invention provides a direct dye-based imaging system to detect AV in cells or tissues. In alternative embodiments, compositions of the invention can quickly and reproducibly detect AV under a range of conditions; thus they can be used to investigate the regulation and physiological/medical relevance of the macroautophagy (or autophagy) intracellular pathway. Thus, in alternative embodiments, compositions of the invention compositions and methods of the invention are used to study the dynamic formation and vesicle flux associated with the de novo biosynthesis and turnover of autophagy.

In alternative embodiments, the compositions of the invention comprise auto-fluorescent compounds, including monodansylcadaverine (MDC) derivatives and fluorescent-conjugated diamine derivatives, and these compositions can be used to detect (e.g., stain), localize and/or measure the amount of AVs or AV sub-populations, including an autophagosome AV sub-population, an autolysosome AV sub-population and/or a lysosomal vesicle AV sub-population.

In alternative embodiments, compounds of the invention are used for a wide variety of physiology-medically relevant applications. For example, in alternative embodiments, compounds and methods of the invention can be used as diagnostic tools to image the induction and flux of AV pathway under a wide range of conditions. In alternative embodiments, compounds and methods of the invention can be used define the role of autophagy in an inherited or an acquired disorder (e.g., a human disorder), including inherited or acquired disorder(s) associated with a lysosomal dysfunction, protein aggregate formation, an infection, a metabolic disorder and/or cellular aging.

In alternative embodiments, compounds of the invention comprise four primary components (or “domains” or moieties): a reporter group, linker bond, linker and reactive head group. These components cooperatively influence the overall properties of these compounds of the invention (which can act as dyes and labels) in biological systems in terms of their selectivity, specificity and stability. For example, in alternative aspects, compounds of the invention selectively label autophagic vesicles (AVs), or selectively label a subset of AVs, wherein the AV subpopulation can comprise an autophagosome AV subpopulation, an autolysosome AV subpopulation and/or a lysosomal vesicle AV subpopulation.

### Fluorophore/Reporter Groups or Domains

The reporter components (or “domains”, e.g., detectable domains, or moieties) of the chimeric compositions of the invention are responsible for the detection and spatial localization in a biological sample. These may be based on, but not restricted to fluorescence in the ultra-violet, visible, infrared spectral regions, or may report via radiofrequencies (MRI/NMR) and well as radioactive detection. In addition, the reporter group may contain heavy atoms for detection using electron microscopy (EM or TEM), scanning EM (SEM) or mass spectral or equivalent techniques. In alternative embodiments, the reporter (domains or moieties) comprise functional groups that either turn off or on its reporting function from its native state, but in the presence of a biological sample (for example; pH change, presence of a specific enzyme, metal etc.) changes its state, giving further details to the biological environment in an autophagic vesicle. For example, in one embodiment, the reporter domain or moiety provides a detectable signal in an acidic environment, e.g., a subcellular vesicle such as a lysosomal vesicle AV subpopulation.

In alternative embodiments, the reporter domain or moiety (e.g., a cadaverine derivative) comprises, or is modified with, a radioactive, a luminescent e.g., bioluminescent, paramagnetic or a fluorescent reagent. In alternative embodiments for in vivo use, the composition of the invention is injected via catheter or systemically and the signal is detected using a detecting device, e.g., a luminometer, attached to a fiberoptic probe that is inserted into the organ and/or tissue via a catheter or a needle or related device.

In alternative embodiments, the reporter domain or moiety (e.g., a cadaverine derivative) comprises, or is modified or derivatized with, a paramagnetic agent (e.g. gadolinium, ferritin) and injected into an organ and/or tissue, or an organism, and the amount of reporter (e.g., paramagnetic agent) incorporated into a particular AV, organ and/or tissue is a reflection of the extent of autophagy in that site, which can be assessed using nuclear magnetic resonance imaging.

In alternative embodiments, the readings, e.g., radioactive, bioluminescent or paramagnetic or fluorescence readings, can be normalized to cell number or total protein or number of cell nuclei.

In one embodiment, compositions and methods, e.g., assays, of this invention can be used with any cell and/or any cell line, organ and/or tissue, and can comprise the use of a fluorescent dye, a radioactive molecule, or a bioluminescent or paramagnetic composition, and a few washing steps, which in some aspects can offer advantage(s) to existing methods.

In alternative embodiments, the cadaverine reagent monodansylcadaverine (MDC) or the related dyes (BODIPY®-TR-cadaverine, or ALEXAFLUOR® 488-cadaverine; Molecular Probes, Invitrogen, Carlsbad, Calif.) are used to practice this invention to label autophagosomes.

In one embodiment, as in an exemplary assay described herein, a biopsy sample can be scored for autophagy within 60 minutes, and can provide a quantitative result that can be normalized to total protein or number of nuclei in the sample. This embodiment is simple and requires minimal expertise.

In one embodiment, as in an exemplary assay described herein, an autophagy dye (BODIPY®-TR-cadaverine) is introduced via specialized catheter, and incorporation of the dye into autophagosomes in assessed by fluorescence measurements using two fiberoptic probes (one for excitation, the other for emission detection) incorporated into the catheter.

The examples described herein validate the use of compositions of this invention (e.g., compositions comprising fluorescent cadaverine, cadaverine derivatives, or equivalents) in the high-throughput assays of this invention, including the plate-based assays of this invention. The examples described herein validate the use of compositions of the invention, including cadaverine derivatives or equivalents, in tissue and/or organ samples, e.g., as described below, from rat or mouse hearts. In alternative embodiments, fluorescent, radioactive, bioluminescent and/or paramagnetic reagents are used.

In alternative embodiments, the cadaverine-derivatized compositions for measuring the amount of autophagic activity in cells or tissues used to practice this invention are commercially available or can be synthesized for specific indications.

In alternative embodiments, any means, such as fluorescence, positron emission tomography (PET) imaging, nuclear magnetic resonance (NMR) imaging, transmission electron microscopy (TEM) and the like can be used to detect the compositions of the invention (e.g., cadaverine-derivatized compositions) and/or to practice the methods of this invention, e.g., in vitro, in situ or in vivo. In one aspect, a fiberoptic catheter is used for in situ and/or in vivo detection of autophagy.

### Fluorophore/Linker Bond

In some embodiments, the bond between the reporter and linker groups may also influence the labeling of autophagic vesicles of compositions of this invention, as well as their stability in a biological sample. The type of bond is dependent on the reporter, linker and reactive head groups.

### Linker Group

In alternative embodiments, the linker group connects the reporter to the reactive head group. In some embodiments, the length of the linker group, as well as the presence of other heteroatoms and functional groups can strongly influence the labeling of autophagic vesicles via the composition of this invention. In some embodiments, the structure of this linker interacts with the membrane. In alternative embodiments the composition and/or the length of the linker group can be modified to optimize use e.g., in a particular desired cell type, for a particular detection moiety and/or a particular use.

### Reactive Head Group

In alternative embodiments, compositions of this invention comprise at least one basic nitrogen group, e.g., when the compositions of this invention are used as autophagic vesicle dyes. Exemplary “basic nitrogen” groups include but are not limited to primary, secondary and tertiary aliphatic amines, aromatic and heteroaromatic amines, guanidines and polyamines. In alternative embodiments, the basic nitrogen can be replaced with an hydrogen.

### Kits

The invention provides kits comprising compositions of the invention, and in alternative embodiments comprise instructions for practicing the methods of the invention, e.g., directions as to indications, amounts to be used, patient populations for practicing the invention and the like.

### Formulations and Possible Routes of Administration

In alternative embodiments, the invention provides pharmaceutical compositions or formulations comprising one or more chimeric molecules of the invention, or a liposome of the invention; or a pharmaceutical composition or formulation of the invention. In alternative embodiments, the pharmaceutical composition or formulation is formulated with a pharmaceutically acceptable excipient, an appropriate buffer and the like, including any additional appropriate additional additive, e.g., such as a preservative or a stabilizer.

In alternative embodiments, the invention provides inhalants or spray formulations comprising any composition of the invention and optionally also a pharmaceutically acceptable excipient, an appropriate buffer and the like.

In alternative embodiments, the invention provides parenteral or enteral formulations. Details on techniques for alternative formulations and administrations that can be used to make compositions of the invention or practice the invention are well described in the scientific and patent literature, see, e.g., the latest edition of Remington's Pharmaceutical Sciences, Maack Publishing Co, Easton Pa. (“Remington's”) (e.g., Remington, The Science and Practice of Pharmacy, 21st Edition, by University of the Sciences in Philadelphia, Editor).

Uses of compositions and formulations of the invention as pharmaceutical compositions include their use as diagnostic agents, e.g., for determining levels of autophagy in a particular cell type, organ and/or tissue. Uses of compositions and formulations of the invention as pharmaceutical compositions include their use for in vivo screening of compounds, e.g., as in experimental animals, or ex vivo, e.g., in perfused organs or tissues ex vivo, to test for compounds that effect AVs or autophagy, as described herein. Uses of compositions and formulations of the invention as pharmaceutical compositions also include their use in assays and screening protocols for characterizing imaging tools, including fluorescent dyes or probes, that are incorporated into the chimeric molecules of the invention. Use of compositions and formulations of the invention as pharmaceutical compositions also includes their use in methods for the screening (e.g., high-throughput screening) of drugs or reagents that modulate autophagy or the amount of autophagosomes (AV) in a cell extract, cell, tissue, organ, organism or individual.

Compositions and formulations of the invention can be made for injectable use, e.g., they can include sterile aqueous solutions or dispersions and sterile powders for the extemporaneous preparation of sterile injectable solutions or dispersions. In alternative embodiments they can be sterile and/or fluid to the extent that easy syringability exists; or can be stable under the conditions of manufacture and storage; or can be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The invention provides oil-based formulations and/or pharmaceuticals for administration of compositions of the invention. Oil-based suspensions can be formulated by suspending an active agent (e.g., a chimeric composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate.

The formulations of the invention can comprise auxiliary substances as required e.g., to approximate physiological conditions such as pH adjusting and buffering agents, toxicity adjusting agents, e.g., sodium acetate, sodium chloride (e.g., saline), potassium chloride, calcium chloride, sodium lactate and the like, or any pharmaceutically acceptable composition.

### High-Throughput Screening

In alternative embodiments, the invention provides methods for the high-throughput screening of drugs or reagents that modulate autophagy or the amount of autophagosomes (AV) in a cell extract, cell, tissue, organ, organism or individual. Large numbers of compounds can be quickly and efficiently tested using “high throughput screening (HTS)” methods. High throughput screening methods can involve providing a library containing a large number of potential (e.g., test or candidate compounds) compounds (e.g., AV-inhibiting, autophagy inhibiting or AV labeling compounds, as described herein). Such “combinatorial chemical libraries” are then screened in one or more assays to identify those library members (particular chemical species or subclasses) that display a desired characteristic activity e.g., AV-inhibiting, autophagy inhibiting or AV labeling.

High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput.

The invention will be further described with reference to the following examples; however, it is to be understood that the invention is not limited to such examples.

### EXAMPLES

### Example 1

### Demonstrating the Efficacy of Compositions of the Invention

The following example demonstrates the efficacy and advantages of compositions and methods of this invention by describing an exemplary high-throughput cadaverine protocol for a plate-reader:

For 24 Well Plates:
Plate 75,000 cells per well
Wait for cells to achieve approximately 80% confluence (approximately 1 day)
Treat cells (3 h 30 min starvation, 3 mL of medium)
Rinse cells with PBS (1 ml)
Incubate cells with probe for 10 minutes at 37° C.
Wash cells four times with PBS (1 mL)
Lyse cells by incubating in 10 mM Tris-Cl pH 8 containing 0.1% Triton X-100 for 20 minutes (500 uL)
Measure fluorescence in plate reader
Add ethidium bromide to a final concentration of 0.2 mM and measure fluorescence (exc=530 nm; em=590 nm)
Normalize results to the number of cells (ethidium bromide reading)

For 35 mm MATTEK™ Dishes:
Use the same protocol, but plate 200,000 cells per dish

Probes:
ALEXAFLUOR488™ cadaverine (A30676, Invitrogen, Carlsbad, Calif.)
   Final concentration 25 uM, exc=493 nm; em=516 nm.
BODIPY TR™ cadaverine (D6251, Invitrogen, Carlsbad, Calif.)
   Final concentration 125 nM, exc=588 nm; em=616 nm.

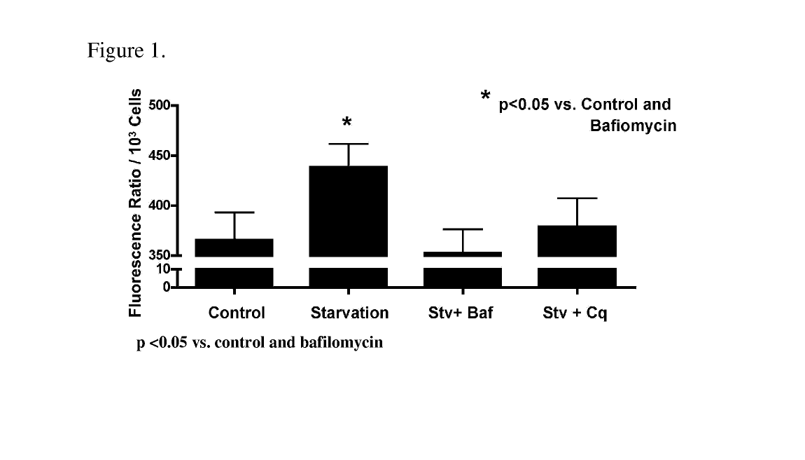

This FIG. 1 shows the relative fluorescence of HL-1 cells loaded with ALEXAFLUOR488™-cadaverine, normalized to cell number (ethidium bromide fluorescence). Starvation increases autophagy (reflected by increased fluorescence), which is partially blocked by CsA, Bafilomycin A1 (Bf), and chloroquine (Cq).

### Exemplary Tissue Autophagosome Quantification Protocol

The invention provides Fluorescent Cadaverine Plate Reader Assays for Quantifying Autophagy in a tissue, for example:

An exemplary Fluorescent Cadaverine Plate Reader Assay for Quantifying Autophagy in a tissue comprises:

### Tissue Preparation

1) Mince 1-5 mm³ tissue sample in 1-2 mL homogenization buffer in 35 mm dish
2) Polytron at ½ speed, 5 sec, on ice in 15 mL round bottom polypropylene tube
3) Spin out nuclei and heavy membranes @ 1000 g, 5 min, 4° C. in 15 mL Falcon tube
4) Move post-nuclear supernatant into 1.5 mL Eppendorf tube
5) Add MDC (or Cadaverine 488) to final concentration of 25 μM
6) Incubate on ice 10 min protected from light
7) Spin sample 20,000×g, 20 min, 4° C.
8) Aspirate supernatant and rinse off pellet with 1 mL cold resuspension buffer 2×'s
9) Resuspend pellet in 350 μL resuspension buffer until mixed evenly
10) Add 100 μL per well in triplicate to black 96-well plate
11) Read on Fluorescence plate reader @ excitation/emission 495 nm/519 nm
12) Use remaining sample to run Bradford assay to quantify protein concentration.

### Homogenization Buffer:

17.1 g Sucrose

2 mL 100 mM Na₂EDTA

0.477 g Hepes Free Acid

Bring volume up to 200 mL with diH₂O

pH 7.0
Add fresh protease inhibitors to 10 mL aliquot prior to use

### Resuspension Buffer:

140 mM KCl

10 mM MgCl₂

10 mM MOPS pH 7.4

5 mM KH₂PO₄

1 mM EGTA
Add fresh protease inhibitors to 10 mL aliquot prior to each use.

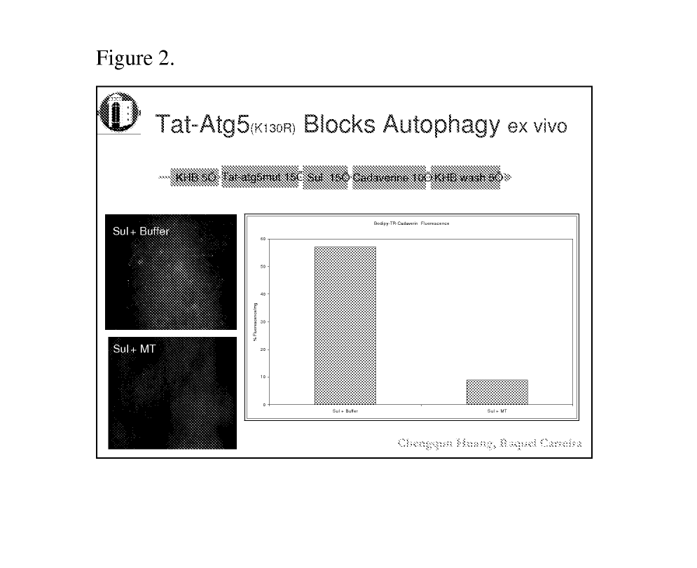

The hearts were perfused with BODIPY-TR™-cadaverine followed by washout, then homogenized and fluorescence read in plate reader. In FIG. 2, the two images at left shows heart tissue loaded with dye, and bar graph shows quantitation of dye incorporation into autophagosomes. Sulfaphenazole (SUL) is a potent inducer of autophagy while Tat-Atg5(K130R) (MT) blocks the formation of autophagosomes. Thus, FIG. 2 upper left image illustrates heart cells induced for autophagy by SUL, and FIG. 2 lower left image illustrates this SUL-induced autophagy blocked by Tat-Atg5(K130R). FIG. 2 right image is a graph illustrating quantitation of dye incorporation into autophagosomes, with the left column graphically quantitating the upper left image's fluorescence and the right column graphically quantitating the lower left image's (the Tat-Atg5(K130R)-inhibited) fluorescence.

In subsequent assays we found dye can be added to a homogenate rather than (or in addition to) perfusing the whole heart. Thus, in one embodiment the methods of the invention are practiced using tissue (e.g., heart) homogenates, in addition to intact organ perfusion.

### Example 2

### Exemplary Assays for Labeling Autophagosomes

The following example demonstrates exemplary assays of the invention for labeling autophagosomes (also called autophagic vesicles, or AV) and for screening for fluorescent dyes or probes that can be incorporated into chimeric molecules used to practice the compositions and methods of this invention. For example, in alternative embodiments, compositions of the invention comprise a first domain or moiety comprising an autophagosome labeling moiety (e.g., an ethylenediamine, a 1,3-diaminopropane, a 1,4-diaminobutane, a 1,6 diaminohexane, a 2,2′ (ethylenedioxy)diethylamine) and a second domain or moiety comprising a detectable composition or moiety. In alternative embodiments, these detectable compositions or moieties comprise fluorescent dyes or probes that label intracellular organelles, including in alternative aspects, labeling components of the autophagic pathway.

In alternative embodiments, the invention provides a direct dye-based imaging system to detect AV in cells or tissues. In alternative embodiments, the invention provides assays and screening protocols for characterizing imaging tools, including fluorescent dyes or probes, that are incorporated into the chimeric molecules of the invention.

In alternative embodiments, fluorescent dyes or probes to be screened are linked or conjugated to a small (18 kDa) ubiquitin-like microtubule-associated light chain 3 (MAP-LC3 or Atg8 protein); a positive control can be an LC3/Atg8 protein linked or conjugated to a Green Fluorescent Protein (GFP), e.g., as a GFP-N-terminal fusion construct with LC3. When expressed in a wide variety of cell types the green fluorescent protein-tagged autophagic marker protein light chain 3 (GFP-LC3) protein shows a diffuse cytoplasmic distribution but with pathway activation it is rapidly recruited to developing autophagosomes. This results in the formation of microscopic puncta that can be readily detected and imaged by fluorescent microscopy or flow cytometry. The extent to which GFP-LC3-II is recruited into punctate structures closely correlates with the level of autophagy and is now widely used as a reliable indicator of autophagic activity within a cell.

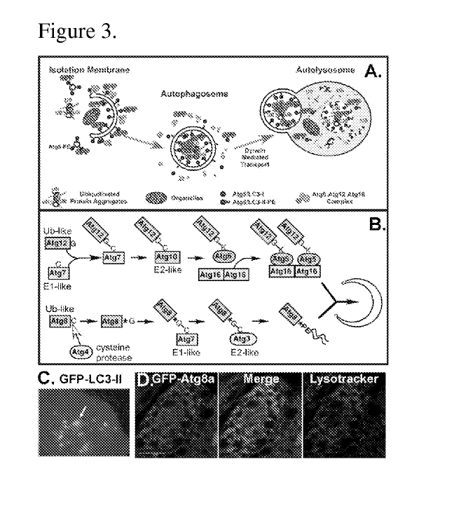

FIG. 3A illustrates an Autophagy Pathway, including AV formation, trafficking and fusion with lysosomes. As illustrated in the figure, a growing phagophore engulfs cytoplasmic material and develops into an autophagosome. Once an AV is mature external proteins are removed and the vesicle is trafficked to and fuses with lysosomes, forming a new autolysosome. GFP-LC3 proteins are used to mark autophagic vesicles. FIG. 3B illustrates Dual ubiquitin-like pathways. With pathway activation the LC3-I protein is processed by the cysteine protease, Atg4. The exposed reactive C-terminal glycine used for conjugation to Atg7 (E1-like), Atg3 (E2-like) and finally to lipids (PE) and forms the hybrid LC3-II molecule. LC3-II becomes an integral part of the growing inner and outer membranes and remains inside the vesicle until it is degraded in the lysosome. FIG. 3C illustrates an image showing GFP-LC3-positive puncta in cardiomyoctyes with up-regulated autophagy. FIG. 3D illustrates three images showing GFP-Atg8a (the left image) and LYSOTRACKER™ (red) stain (the right image) (and a merged image, the middle image) overlapping vesicles at a developmental period when autophagy is under hormonal control and up regulated in flies. These cells are undergoing apoptosis.

Additional transgenic constructs have been developed consisting of different fluorescent tags (mCherry) or other autophagic components (GFP-Atg5) and are being used to characterize additional features of the pathway. However, once autophagosome formation is complete most surface proteins “de-coat” and no longer mark AV, thus making constructs like GFP-Atg5 a less attractive tool. Furthermore, data suggests that enhanced expression of pathway components may alter endogenous regulations of autophagy (see e.g., Simonsen (2008) Autophagy 4:176-184).

Transmission electron microscopy (TEM) can image the double-membrane structure of AV; however, it is technically demanding and requires a significant level of expertise and time, limiting the number of samples that can be analyzed for a given experiment. Also coupling TEM imaging with immunocytochemisty for protein/structural co-localization studies (e.g., gold-conjugated 2ndary antibodies) is an exceptionally difficult technique, only preformed by people well acquainted with the procedure. The use of fluorescent-tagged expression constructs, like GFP-LC3 has greatly simplified the detection and imaging of autophagosomes to the point where it is routinely used to detect and quantify AV formation and pathway flux in living or fixed samples (e.g., 3.5% formaldehyde). The main draw back for this method is the development, transfection or infection of expression constructs into cultured cells. A concern is that expression of the fusion proteins may alter the endogenous autophagy levels or pathway flux.

The Drosophila model system can be used to screen for the efficacy of composition of this invention (key features of the autophagic pathway have been characterized using the Drosophila model system, where genetic alterations to autophagy are well documented). Imaging the dynamic flux of autophagosome formation and turnover can be done using larval fat body tissues. The pathway can be quickly induced using traditional methods like amino acid withdrawal (fasting). It is also under hormonal control (e.g., ecdysone) and shows extensive induction in most larval tissues as part of a programmed cell death pathway. As a result staged fat body tissues (homogeneous with large cells) from 3^{rd} instar larvae can be easily collected and used for direct in vivo examination of autophagy.

Drosophila transgenic tools can be used to screen for the efficacy of composition of this invention; these tools together with the dipartite GAL4/UAS expression system can be used to study autophagic dynamics and vesicle formation, see FIG. 3C. LYSOTRACKER™ (Invitrogen, Carlsbad, Calif.), which stains acidified organelles including lysosomes, late multi-vesicular endosomes and autolysosomes, has also been extensively used in this system. Both LYSOTRACKER™ Red and GFP-dAtg8a (green) show tight co-localization and specifically highlighting mature autolysosome vesicles in 3^{rd} instar fat body cells, see FIG. 3D.

Labeling Autophagosomes in Cardiac Myocytes. In one embodiment, an mCherry-LC3 fusion protein can be used to evaluate the efficacy of a composition of this invention to detect autophagy. To better examine autophagy in cardiomyoctes, transgenic mouse lines that express the mCherry-LC3 fusion protein in the heart were generated (α-myosin heavy chain promoter, cardiac-restricted). In this genetic background endothelial and fibroblast cells do not express mCherry-LC3, eliminating confusion with the study of autophagy in cardiomyocytes. Cherry-LC3 also has several advantages over GFP-LC3. It retains its fluorescence in acidified lysosomes, and there is minimal background auto-fluorescence in cardiac tissue.

Characterization of the αMCH-mCherry-LC3 mice indicates no apparent effects on cardiac function, and marks AV as expected. Images of heart tissue from fed and 48 hr-starved mice reveals there is a substantial increase in the number of autophagosomal vesicles, consistent with increased autophagy. Isolated mCherry-LC3 hearts were subjected to global ischemia (30 min), or ischemia (30 min) and 1 hr reperfusion on a Langendorff setup, and performed in vivo ischemia/reperfusion. Cryosections from these hearts reveal an increase in the abundance of fluorescent puncta, indicative of an increase in AVs.

Direct labeling of autophagosomes. While mCherry- and GFP-LC3 mice and GFP-Atg8a flies are valuable screening and research tools, non-transgenic methods also can be used to measure autophagy and the efficacy of compositions of this invention. Thus, in one embodiment, a monodansylcadaverine (MDC) compound was used. MDC is known to label acidified vesicle sub-populations like late endosomes, lysosomes, and autophagosomes, see e.g., Iwai-Kanai (2008) Autophagy 4:322-329; Perry (2009) Methods Enzymol 453:325-342.

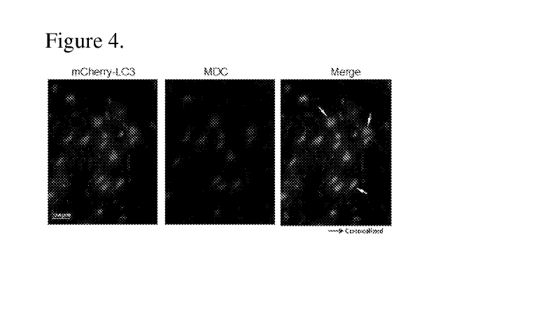

To examine its labeling profile in cardiac tissues, mCherry-LC3 mice were injected with MDC (1.5 mg/kg i.p.) 1 hr before being sacrificed, e.g., see Iwai-Kanai (2008) supra, Yitzhaki (2009) Basic Res. Cardiol. 104:157-167. Hearts were collected and frozen tissues sections prepared for imaging. Under conditions where autophagy is activated and mCherry-LC3 puncta formed, MDC-labeled structures were similarly up regulated, see FIG. 4, and see e.g. Iwai-Kanai (2008) supra. MDC labeled a subset of mCherry-LC3-positive structures, presumably fused autolysosome vesicles. An instance of MDC labeled structures that were not positive for mCherry-LC3 was not detected, demonstrating that MDC is a specific and suitable reagent for the in vivo assessment of autophagy. While others have found MDC to be non-specific, under these conditions the compound shows excellent co-localization with mCherry-LC3 puncta, see FIG. 4, and see e.g. Iwai-Kanai (2008) supra.

FIG. 4 illustrates three images, including mCherry and MDC labeling of vesicles. mCherry-LC3 expressing mice were treated with rapamycin and hearts prepared for fluorescent imaging. As illustrated in the middle image of FIG. 4, MDC shows a significant level of co-localization with mCherry-LC3 positive puncta (arrows in the right image). As illustrated in the left image of FIG. 4, mCherry-LC3-II highlights a subset of structures not stained by MDC, suggesting MDC labels acidified autolysosomes and lysosomes. The right image of FIG. 4 is a merge of the mCherry-LC3 and MDC images.

Initial synthesis of autophagic specific dyes of this invention was based on MDC. While the MDC staining of AV shows considerable promise with fresh cell or tissue preparations, the compound has its limitations. While MDC does show significant photobleaching following normal fluorescence exposure, it has stability issues during storage and cannot be used on fixed samples. In alternative embodiments, dyes used in compositions of this invention are vesicle selective, have multiple fluorescent excitation/emission spectra and can be used for several imaging applications. Thus, the new dyes of this invention will greatly benefit autophagy research.

The design of the initial autophagic vesicle dyes was based on the known structural properties of MDC. Fluorescein (FITC) was selected as the initial fluorophore because it is widely used in biological systems, is membrane permeable, has low cellular toxicity and has emission spectra that are useful with most imaging systems. The distance between the terminus amine and fluorescein group is anticipated to affect the labeling of acidic vesicles. Niemann (2001) J. Histochem. Cytochem. 49: 177-185, attributed the staining of AV with MDC to ion-trapping and interaction with the autophagic vesicle membrane lipids. An optimal effect was found for the five carbon compounds.

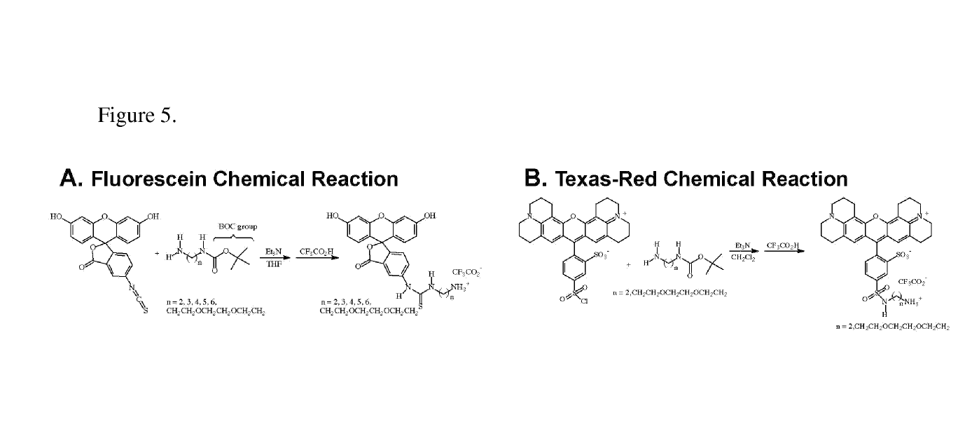

Therefore, to examine ion trapping and lipid membrane interaction effects, a series of linear mono-BOC protected diamines, C₂-C₆ and 3,6-dioxa-1,8-octanediamine, were used to generate six new fluorescein-conjugated molecules, see FIG. 5A. The pentanediamine, cadaverine, was bracketed in the middle of this set of compounds, and was expected to show similar results to those of Niemann (2001) supra. The amines were coupled with fluorescein isothiocyanate in the presence of triethylamine.

FIG. 5 illustrates an exemplary synthesis of Fluorescein- (FIG. 5A) and Texas Red- (FIG. 5B) conjugated Dyes for use in practicing this invention: the BOC-protected group was then removed with trifluoroacetic acid and the dye purified by selective precipitation on addition of diethyl ether to the methanol solution of the reaction product, as described e.g., in Lorand (1983) Ann. N.Y. Acad. Sci. 421:10-27. Characterization by proton NMR spectroscopy gave acceptable spectra in agreement with expected values. Analysis by electrospray (ESI) mass spectroscopy gave an ion with the expected molecular weight.

Texas Red was chosen as the second fluorophore, since it is a commonly used dye and has emission spectrum that is shifted to longer red wavelengths (approximately 615 nm). As a consequence, it generates little background fluorescence and has minimal overlap or bleed-through with fluorescein dyes or GFP. The conjugates are photo stable and bright. We prepared derivatives of the Texas Red sulfonyl chloride with the two linkers found most effective in the fluorescein study. The mono-BOC protected ethylene diamine and the 3,6-dioxa-1,8-octanediamine compounds were reacted with sulfonyl chloride in the presence of a trialkylamine and the protecting group removed with trifluoroacetic acid, see FIG. 5B. Solvent evaporation gave a relatively pure product. Analysis by ESI mass spectrometry gave the expected molecular ion.

Staining of Drosophila Tissues. To perform a rapid first-pass examination of the compounds ability to stain AV we examined fat body tissues from wandering 3^{rd} instar Drosophila larvae. 1 mM DMSO stock solutions were prepared for each compound and stored at −20° C. Fat body tissue from wild type fly larvae were dissected from the surrounding cuticle and organs, and placed in 1 ml iced PBS solution. Tissues were immediately stained for 10 min, in a final 10 μM concentration for each dye. Samples were rinsed twice with 1×PBS, mounted with VECTASHIELD™ (Vector Laboratories, Inc, Burlingame, Calif.) and immediately imaged using a scanning confocal fluorescent microscope (Leica, FITC channel). As a positive control, fly tissues (3^{rd} instar) were also stained with BODIPY-TexasRed-cadaverine (Invitrogen, red).

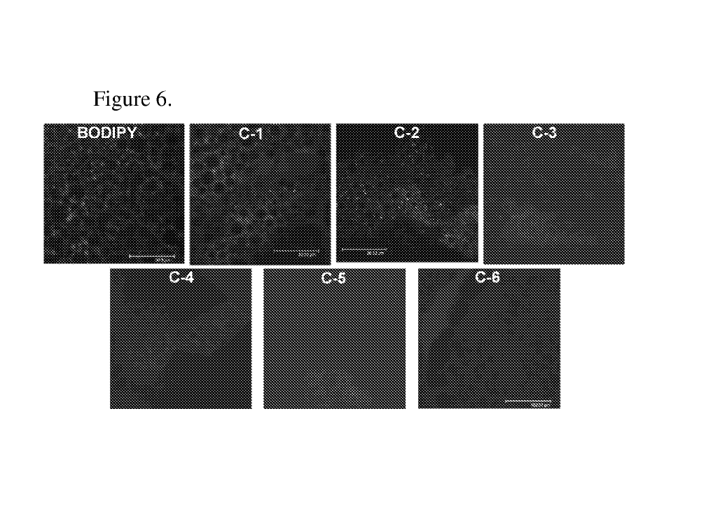

As seen in the images illustrated in FIG. 6, abundant BODIPY-labeled puncta are detected throughout the larval fat body. This labeling is consistent with the extensive levels of autophagy that naturally occur in this tissue at this developmental time point. Under the same conditions the exemplary FITC-ET-C1 and FITC-TG-C2 compounds (see FIG. 5A and discussion above) also show the clear staining of cytoplasmic puncta, consistent with AV labeling.

FIG. 6: Fly tissues with activated autophagy were collected and individually stained for 10 min with one of seven dyes. BODIPY-cadaverine was included as a positive control. The C-3, C-4, C-5 and C-6 dyes did not mark intracellular vesicles in fresh tissue preparations. The BODIPY-dye shows a robust staining pattern, as do the exemplary C-1 (FITC-ET) and C-2 (FITC-TG) compounds. Compounds also can be tested in fixed tissues.

Additional studies of larval fat body tissue focused on the co-localization of LYSOTRACKER™ Red with the FITC-ET and FITC-TG compounds. To generate a different cellular composition of AVs, young 2^{nd} instar larvae were collected and placed on sucrose-only culturing media for 3 hrs (amino acid starvation). The fat body tissue was dissected on ice and stained with LYSOTRACKER™ Red (Invitrogen) and the FITC-ET or FITC-TG (green) compounds, rinsed in PBS and immediately confocal imaged. When deprived of amino acids Drosophila quickly up regulate the pathway and produce numerous new AV. Previous Drosophila studies have shown LYSOTRACKER™ Red highlights both autolysosomes and lysosomes, see e.g., Grewal S S. Insulin/TOR signaling in growth and homeostasis: A view from the fly world. Int. J. Biochem. Cell. Biol, 2008; Rusten (2004) Dev. Cell 7:179-192; Sebastia (2006) J. Neural. Transm. 113:1837-1845.

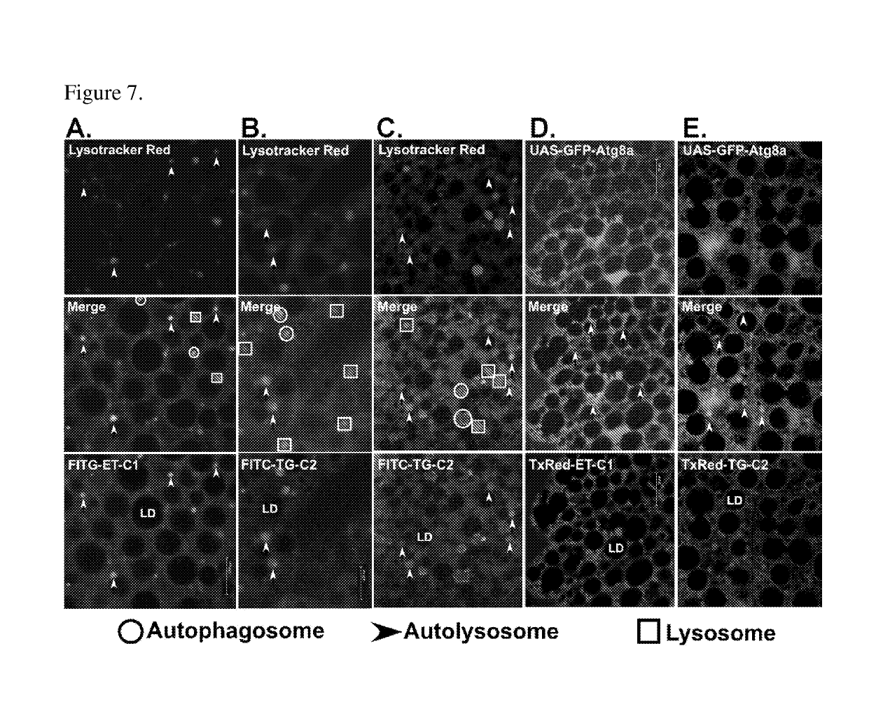

In this experiment both compounds stained a significant number of puncta following amino acid deprivation (FIG. 7 A-B). From these double labeling experiments, three distinct vesicle sub-populations can be detected that include FITC+ (green, circle), LYSOTRACKER™+ (red, squares) and double-labeled vesicles (yellow, arrows). This indicates FITC-ET, FITC-TG and LYSOTRACKER™ selectively partition into distinct vesicle populations both FITC-dyes are detecting AV and may not be partitioning into vesicles due to their internal pH.

Labeling was also repeated with tissue undergoing programmed cell death and a similar pattern of staining was found with LYSOTRACKER™ Red and the FITC-TG dye, see FIG. 7C. These studies indicates the FITC-dyes highlight a population of vesicles that are distinct from LYSOTRACKER™ and could be used to study the early in vivo formation, maturation and fusion events of AV within cells. To further confirm the specificity of the FITC-TG AV staining, fat body tissue was prepared from Atg1−/− larvae and compared with wild type controls.

FIG. 7A-B: Fly larvae were fasted and fat body tissues stained with LYSOTRACKER™ and FITC-ET (FIG. 7A) or FITC-TG (FIG. 7B). Both dyes overlap with LYSOTRACKER™ but also detect a unique vesicle population. FIG. 7C: Tissue from larvae undergoing hormone-triggered autophagy was collected and stained with LYSOTRACKER™ and FITC-TG and similar staining was detected. FIG. 7D-E: Fat from larvae expression GFP-Atg8a was collected, fixed (3.5% formaldehyde, PBS) and stained with Texas Red-ET (FIG. 7D) and Texas Red-TG (FIG. 7E). Both dyes show co-localization with GFP-Atg8a. LD=lipid droplet.

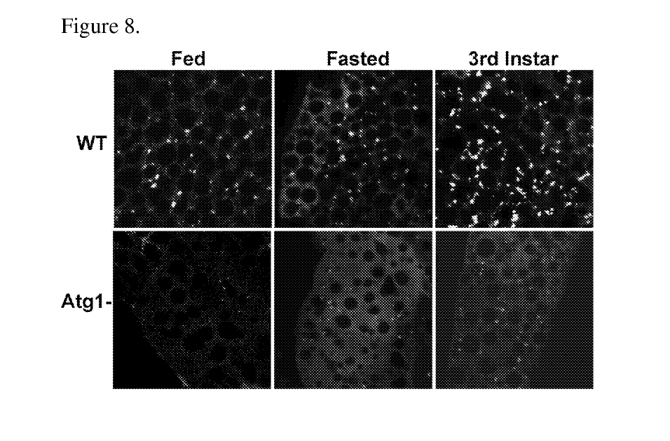

Signaling of the Atg1 protein kinase is essential for pathway induction and AV formation, see e.g., (21, 57). In Drosophila loss-of-function mutations in this gene result in late pupal lethality but have a minor impairment on early development, thus providing sufficient material for imaging studies. As seen previously, both starvation and hormone-dependent induction of the pathway in wild type flies results in significant AV staining; WT, FIG. 8. In contrast, Atg1^{-/-} flies show little or no green FITC-TG positive vesicles for either condition but have some LYSOTRACKER™ positive staining (Atg1−, FIG. 6). This staining pattern is consistent with lysosomes maturing from the endosomal pathway but AV failing to be formed under normal physiological conditions.

FIG. 8 illustrates images of stained tissues from: Fed (left column images), fasted (middle column images) and hormone-induced (“3^{rd} instar” right column images) autophagy profiles in wildtype (upper row images) and Atg1−/− mutant (lower row images) cells. Fat body tissue from larvae that were fed, fasted or undergoing hormone-induced autophagy were collected and stained with LYSOTRACKER™ (red) and the exemplary FITC-TG (green). Even under fed conditions WT larval tissues show basal levels of the pathway. The number of AV vesicles (green, yellow) increases when the pathway is up regulated. In Atg1−/− mutant flies formation of new autophagosomes is inhibited. While LYSOTRACKER™ puncta are detected (red) in these Atg1^{-/-} mutant tissues, the exemplary FITC-TG dye fails to stain autophagosomes or autolysosomes.

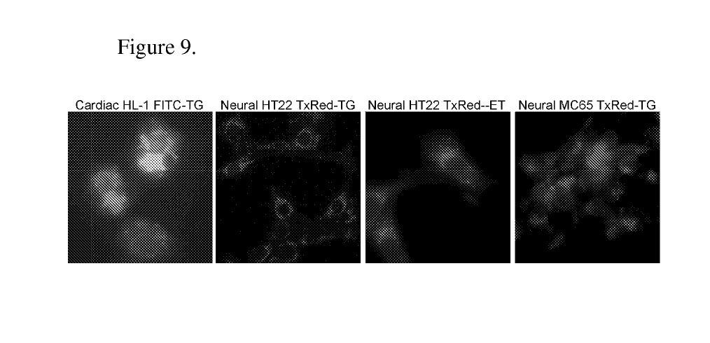

FIG. 9 illustrates the images of the labeling of mouse and human tissue culture cells with exemplary dyes of the invention, as indicated in the figure images: left image is cardiac HL-1 cells stained with FITC-TG label; next image is neural HT-22 cells with Tx-red-TG label; next image is neural HT-22 cells with Tx-red-ET label; right image is neural MC-65 cells with Tx-red-TG label, as discussed below. Mouse HL-1 cells (cardiomyocytes) were fasted for 3 hrs and stained with the exemplary FITC-TG and DAPI. Numerous green puncta were observed. Neural HT22 (mouse) and MC65 (human) cells were fixed for 10 min in 3.5% formaldehyde and stained with TxRed-ET or TxRed-TG. HT22 cells did not receive treatment to activate autophagy but both dyes highlighted numerous puncta, consistent with high levels of basal autophagy in neurons. TxRed-TG stains dense perinuclear structures in MC65 cells, which are expressing Aβ-peptide and forming cytoplasmic aggregates.

Texas-Red compounds and staining of fly tissues and mammalian cells. Based on the preliminary findings and staining patterns of the FITC-ET and FITC-TG compounds, we produced additional dyes using the same amine groups and a different fluorophore head group. For this chemical synthesis two new dyes were produced using the Texas Red fluorophore, assayed for purity and called Texas Red-ET and Texas Red-TG. Initially, both dyes were used at 10 microM working concentration to label AV in Drosophila fat body tissues. An unexpected finding was the Texas Red compounds do not label cytoplasmic vesicle populations in fresh tissue preparations (data not shown) but do highlight puncta in samples that have first been fixed in 3.5% formaldehyde (see FIGS. 7D-E). When compared to the vesicles highlighted in flies expressing the GFP-Atg8a fusion protein, both the Texas Red-ET and Texas Red-TG compounds showed considerable overlap with an autolysosome and lysosomal organelle sub-sets. A second unexpected finding from these studies was that the green FITC-ET and FITC-TG dyes gave the opposite results and did not selectively stain any cellular structure prepared from fixed tissues.

As part of characterizing these novel compounds we also examined cultured cells. HL-1 cardiomyocytes were deprived of amino acids and serum for 3 hrs and then labeled with FITC-TG (green) and the nuclear dye DAPI and imaged using standard fluorescent microscopy (blue, FIG. 9). Green, FITC-TG positive puncta were detected throughout the cytoplasm and near the nucleus.

Neural cells were also examined. Fixed HT22 (mouse hippocampal) and MC65 (human neuroblastoma) cells showed considerable vesicular labeling with both the Texas Red-ET and Texas Red-TG dyes (see FIG. 9). AV labeling of the MC65 cells is of particular interest since the cells produce the neurotoxic Aβ-peptide using a Tet-off expression system (CT-100-hAPP). The Aβ-peptide significantly contributes to the neuropathology and protein aggregates or plaques associated with Alzheimer's disease (FIG. 9).

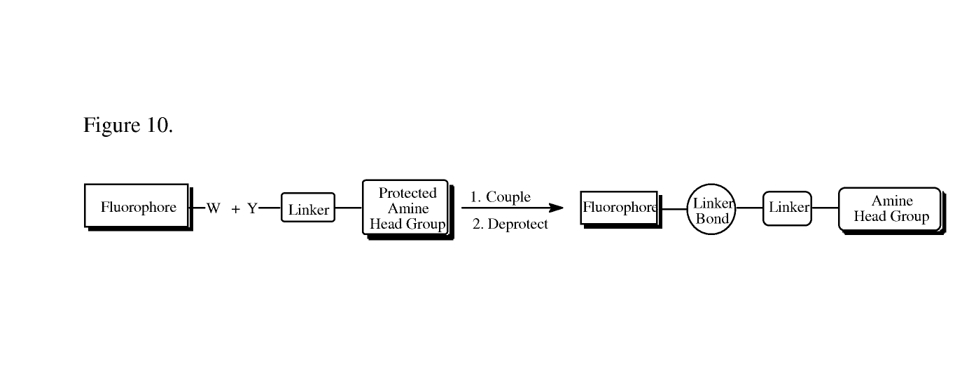

An exemplary synthesis procedure for making fluorescent compounds of the invention is shown in FIG. 10. In one embodiment, compositions of the invention can be divided into four sections, or domains, that can be independently varied to enhance their targeting specificity. In this embodiment, this requires coupling of: 1) a reactive fluorophore with 2) compounds that have a reactive head group (e.g., in one embodiment, an amine or an amine-comprising composition), 3) a variable length linker and 4) a Y-group that forms the other half of the linker bond.

In alternative embodiments of series of exemplary compounds of the invention, three (of the four) of these “sections” or domains will be held constant and the fourth varied. In alternative embodiments, reactive fluorophore compounds are based on known dyes that selectively localize in AVs. The length of the linker group, its chemical type, the reactive head group and its functional group-type are varied.

Each new series of compounds will be assayed, e.g., using a protocol or method as described herein, to determine the selectivity of individual exemplary compounds of the invention for vesicle targeting, selectivity and working concentrations that give optimal staining with minimal background fluorescence. In alternative embodiments, results from in vivo staining patterns are compared with structural information and used to redesign the next cycle of chemical modifications. In alternative embodiments, a different “section” or domain is systemically varied to determine its effects on AV targeting and use as suitable labeling reagent.

FIG. 10. Chemical reaction strategy to generate exemplary fluorescent dyes and compounds of the invention.

The choice of the fluorophore largely controls the absorption and emission wavelengths, but other considerations include the capability of microscope instrumentation and the type of filter sets and excitation source. These features may limit the types of experiments. For these exemplary sections (or domains) three dyes were selected. They have different spectral regions but are used wide used in variety of imaging applications. Fluorescein was initially chosen due to its wide use, relatively high absorption, excellent quantum yield, good stability and low cost. However, it does have a broad emission spectrum that can limit its use in multicolor double-labeling experiments. Photobleaching and a decreased fluorescence below pH 7.0 (pKₐ=6.4) are additional limitations with this compound, thus limiting its application with acidic vesicles (lysosomes). Difluoro-fluorescein (Oregon Green 488) is the fluorinated analog of fluorescein and has the same absorption and emission spectra. It has a lower 4.7 pKₐ, is a useful pH indicator for acidic vesicles and has excellent photostability (similar to ALEXAFLUOR™).

In alternative embodiments of the invention, fluorescein isothiocyanate is used as the “detectable composition or moiety” domain, and the linker length and type of nitrogen head group is varied (e.g., ethylenediamine, 1,3-diaminopropane, 1,4-diaminobutane, 1,6 diaminohexane, 2,2′ (ethylenedioxy)diethylamine and the like). Dyes that show selective AV labeling will also be tested for photostability and pH sensitivity. If photobleaching or high acidity limits application of the fluorescein dye for any particular exemplary composition of the invention, the fluorescein can be substituted with Oregon Green 488.

In embodiment, Texas Red is the fluorophore, or the “detectable composition or moiety” domain. It has an emission spectrum at approximately 615 nm. As a consequence, it has little background and minimal overlap with fluorescein dyes. Texas Red fluorescence is stable between pH 4 to 10 and generates a bright and photostable conjugate. Compositions of the invention comprising Texas Red dyes can be used in the same in vivo assays as exemplary compositions of the invention comprising fluorescein dyes.

In one embodiment, a BODIPY dye is the 3^{rd} fluorophore, or the “detectable composition or moiety” domain. It can have spectral characteristics including; high extinction coefficients, excellent quantum yields, and narrow emission spectral widths allowing multicolor experiments spanning both the visible and infrared spectrum. In general, this family of dyes is resistant to photobleaching. The neutral charge and low molecular weight of BODIPY dyes allow for greater cellular permeability. There are many known structural variations of the BODIPY dyes allowing modification of their spectral properties, e.g., as described by Loudet (2007) Chem. Rev. 107:4891-4932; Ulrich (2008) Angew Chem. Int. Ed. Engl. 47:1184-1201.

In alternative embodiments, the linker group connecting a fluorophore to the “head group”, or the amine-comprising group or domain (e.g., ethylenediamine, 1,3-diaminopropane, 1,4-diaminobutane, 1,6 diaminohexane, 2,2′ (ethylenedioxy)diethylamine), may also affect the overall selectivity of a given dye. Niemann (2001) supra, described that replacing the terminal amino group in MDC with an hydrogen also allowed selective labeling of AV. Niemann (2001) supra, concluded that dyes containing an uncharged group or a protonate-competent amine that could form a positively charged species. This feature may result in greater interactions with the AV double lipid bilayers than negatively charged groups. Niemann (2001) supra found monodansyl derivatives based on 1-alkyl amines do not use an ion-trapping protonation mechanism.

A series of monodansyl compounds were prepared from n-alkyl amines, varying in length from two to eight carbons showed the same vesicle localization pattern as the MDC, with varying fluorescence. Results showed a similar linker group effect (similar to Niemann (2001) supra). The mono-BOC series of FITC dyes gave variable in vivo AV labeling, with the C₂ and triethylene glycol diamine dyes showing excellent AV labeling. AV dyes may operate by two factors; amine group ion trapping and the interaction of the linker with the unique double lipid bilayer structure.

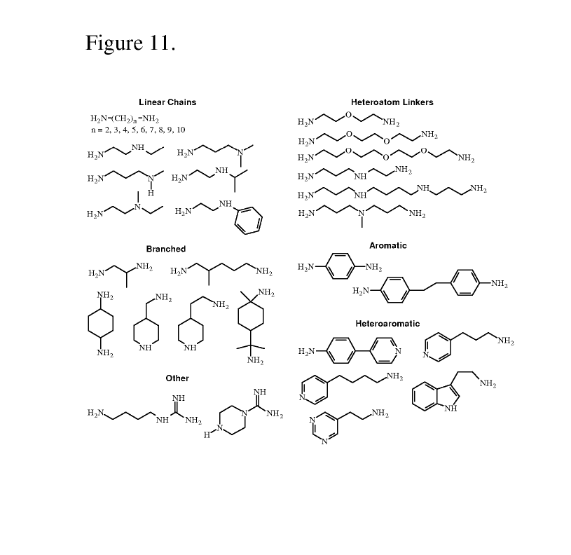

In alternative embodiments, different linkers of varying length, including the presence or absence of heteroatoms, branching, and unsaturated groups for exemplary chimeric compositions of the invention. In alternative embodiments, linker groups comprise commercially available diamines or mono protected diamine compounds. Examples of the linker groups and the amines or heteroaromatic amines are shown in FIG. 11, which illustrates exemplary (representative) examples of commercially available linker and diamine head groups that can be used in compounds of this invention. A diverse series of compounds may be incorporated into fluorophore synthesis systems. Potentially both head groups and linker chains may play a significant role in the selectivity or partitioning of various dyes into different vesicle sub-populations. Critical vesicle characteristics may include: 1) Lipid composition, 2) Membrane structure, 3) Associated proteins, 4) Internal pH; these may ultimately influence the selectivity and specificity of the novel compounds.

In alternative embodiments, alternative linker groups that have enhanced lipid interactions can be used.

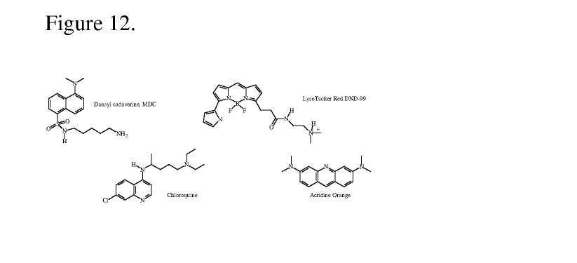

In one embodiment, a chimeric composition of the invention comprises a domain comprising a basic nitrogen head group, e.g., ethylenediamine, 1,3-diaminopropane, 1,4-diaminobutane, 1,6 diaminohexane, 2,2′ (ethylenedioxy)diethylamine, triethylene glycol diamine, or equivalents thereof. Exemplary dyes of this invention used to stain lysosomes and some AV sub-types can comprise a basic nitrogen group. Exemplary chimeric compositions of this invention can comprise an MDC, a LYSOTRACKER RED DND 99™, chloroquine, acridine orange or equivalent, and in alternative embodiments comprise primary, tertiary (N,N-dimethyl or diethyl, aliphatic amines) or N,N-dimethylaniline derivatives, see FIG. 12. While the invention is not limited by any particular mechanism of action, the basic property of the nitrogen group appears to be mechanistically responsible for ion-trapping into acidic vesicles and the different groups may allow dye discrimination due to subtle pH differences. FIG. 12 illustrates exemplary compounds of this invention that preferentially label acidified organelles, including lysosomes and autophagosome; late multi-vesicular endosomes may also be detected by these dyes:

In alternative embodiments, the invention comprises first domain or moiety comprising: a primary amine; a tertiary amine; an N,N-dimethyl or diethyl amine; an aliphatic amine; a heteroaromatic amine, an ethylenediamine, a 1,3-diaminopropane, a 1,4-diaminobutane, a 1,6 diaminohexane, a 2,2′ (ethylenedioxy)diethylamine, a triethylene glycol diamine, an N,N-dimethylaniline, or derivatives or equivalents thereof. In one aspect, guanidine is used; it a more basic compound and should become protonated in less acidic vesicles (autolysosomes), while aliphatic amines, aromatic amines (anilines) and heteroaromatic nitrogen compounds are less basic and may preferentially ion-trap into highly acidic vesicles (lysosomes). However, differences in labeling properties may also be due to steric effects.

While the invention is not limited by any particular mechanism of action, another physical characteristic that can favor localization in acidic vesicles is the chemical properties of polyamines. This includes spermine or a spermidine (linear) as well as bifurcated di- and triamines that have multiple sites for protonation. Unless the head group is a tertiary or heteroaromatic amine, dye synthesis requires protection of the head group during the coupling step, followed by the removal to free the amine group. This chemistry is well established and is expected to proceed with few problems in the formation of protected amines, coupling or de-protection steps. Many of these amines can be purchased directly as BOC compounds or as other protected amines, or prepared as described in Lee (2007) Selective Mono-BOC Protection of Diamines Synthetic Communications 37:747-742.

Optimizing fluorescent linker bonds. The choice of the bond between the dye and linker group is perhaps the least understood with respect to the effectiveness and selectivity of AV labeling of a compound of this invention. In alternative embodiments, linker bonds are chosen to be more resistant to hydrolysis and/or to be stable in an acidic vesicle. If hydrolysis does occur, a loss of signal should be observed even without exposure to fluorescent light. This is differentiated from photobleaching, which only occurs in the presence of irradiation.

In alternative embodiments, linker bonds used in compositions of the invention comprise thioureas, sulfonamides and amides, in their approximate order of stability. However, while the invention is not limited by any particular mechanism of action, it is unknown whether the linker bond influences the selectivity of a dye. This can be explored by comparing several of the exemplary dyes to dyes changed to another linker group; for example, by substituting a thiourea for an amide bond.

Alternative exemplary dyes can be screened using multiple preparation techniques and cell and tissue types, including Drosophila fat body cells, e.g., as described herein. This tissue not only undergoes two types of programmed autophagy and is easily prepared; there also is a wide range of genetic (e.g., Atg1, Atg8a mutants) and transgenic tools available to identify AV staining (e.g., GFP-Atg8a); e.g., assays used to confirm activity of chimeric compositions of the invention can comprise methods and protocols described in e.g. Rusten (2004) supra; Scott (2004) Dev. Cell. 7:167-178; Simonsen (2008) supra. Cellular/tissue permeability and working concentrations needed for optimal staining can be assessed using fresh tissue preparations. Most fresh tissue preparations will include counter-staining with LYSOTRACKER™ Red or Green (Invitrogen). The question of fresh versus fixed preparation for optimal staining also can be addressed in fat cells and imaged using conventional or confocal fluorescent microscopy.

After screening Drosophila tissues, an exemplary dye's staining patterns can be characterized in mammalian cells and/or whole tissue preparations. Exemplary dyes can be examined on both fresh and fixed samples (see e.g., Gaullier (1999) Biochem. Soc. Trans. 27:666-670) following standard techniques and imaged using standard or confocal fluorescent microscopy. During these studies additional fixation techniques can be examined that include first staining biological samples with the compounds then followed by fixation in 4% PFA. Staining patterns can be examined using methanol fixed tissues. Once a particular dye is found to highlight AV in fixed samples, then its staining pattern with paraffin embedded tissues can be tested.

In one aspect, the compatibility of dyes in alternative exemplary compositions of the invention are characterized with immunocytochemistry (ICH) imaging techniques. To fully exploit the use of alternative dyes, their compatibility can be determined with fluorescent antigen-antibody ICH imaging methods. This technique is widely used for cell imaging studies and is indispensable in detecting complex interactions between proteins and individual organelles. IHC involves localizing proteins by exploiting the specific antigen binding of primary antibodies (e.g., acting as unique or specific biomarkers) and is widely used to diagnose cellular abnormalities associated with cancerous tumors, neurological disorders or cardiac defects; alternative protocols that can be used are described in e.g., Finley (2003) J. Neurosci. 23:1254-1264; Hoyer-Hansen (2007) Autophagy 3:381-383; Simonsen (2004) J. Cell. Sci. 117:4239-4251; Simonsen (2008) supra; Simonsen (2007) Autophagy 3:499-501.

The one technical concern is that most samples are typically fixed in 4% PFA (para-formaldehyde) and that detergent permeabilization is needed to allow full access of primary and fluorescent secondary antibodies intracellular components. Initially new AV dyes will be fixed in 4% PFA, PBS and permeabilized with 0.5% Triton-X100, PBS (TBS) for 5-10 min, at RT (standard method). This is generally considered “harsh” treatment of the samples and may not be optimal for preserving critical lipid structure associated with vesicles. Additional fixation (100% methanol, 5 min) and permeabilization (0.05-0.1% Saponin in TBS, 5-20 min, mild) techniques can be examined. The timing of primary and secondary antibody incubations can follow established protocols for a given sample type and images will be collected using confocal microscopy.

In one embodiment, dyes used in a composition of the invention are optimized for use with a plate reader assay for quantitative measurement of AV. In one aspect, a high-throughput protocol based on an MCD compound is used to measure AV levels in samples prepared from cultured cells or tissues, e.g. as described in Perry (2009) Methods Enzymol. 453: 325-342.

For cultured cells, approximately 75,000 cells/well will be plated into 24-well TC plates and grown to approximately 80% confluence (1 day). Autophagy can be induced either by drug treatment (rapamycin) or with starvation (3 to 3.5 hrs in starvation medium). Cells can be rinsed with PBS and incubated with different fluorescent probes for 10 minutes at 37° C. Washed cells can then be incubated in lysis buffer at RT for 20 min (500 μl, 10 mM Tris-Cl pH 8.0, 0.1% TritonX-100). Plates can be read using a microplate spectrophotometer (e.g., by Molecular Devices, Sunnyvale, Calif.), using e.g. SPECTRAMAXPLUS™, SOFTMAX PRO™ software) and individual fluorescence levels detected at the fluorphore appropriate wavelength. To normalize for cell number, ethidium bromide (EB, 0.2 mM final) can be added to each well and fluorescence measured (exc=530 nm; em=590 nm). Fluorescence levels for each dye can be normalized to the number of cells (EB, reading), e.g., as described by Perry (2009) supra.

In alternative embodiments, exemplary compositions comprising cadaverine-based dyes are screened using a plate-reader technique to measure autophagy in fresh or frozen tissue samples. Mice as test subjects can be treated/screened with a variety of compounds or physiological conditions (e.g., caloric restriction, coronary ischemia), following predefined protocols. Tissue (1-5 mm³) can be minced in 1 to 2 ml homogenization buffer (e.g., 250 mM sucrose, 1 mM Na₂EDTA, 10 mM Hepes Free Acid, final pH 7.0) and further disrupted using a polytron (on ice, ½ speed, 5 sec), e.g., as described in Perry (2009) supra. Heavy membranes and nuclei can be pelleted by centrifugation at 1000×g at 4° C. for 5 min Duplicate aliquots of the post-nuclear supernatant can be placed into fresh 1.5 ml Eppendorf tubes and the remaining pellet saved on ice.

In one exemplary protocol, ALEXA FLUOR CADAVERINE 488™ (5 mM stock, Molecular Probes) is added to the supernatant to a final 25 μM concentration, followed by a 10 min, iced incubation. The nuclear pellet can be placed in a resuspension buffer containing the HOECSHT 33342™ nuclear dye (Invitrogen, Life Technologies, Carlsbad, Calif.) and rotated at 4° C. for 10 min Cadaverine labeled samples can be spun at 20,000×g for 20 min at 4° C. and the nuclear pellet at 1,000×g for 5 min at 4° C. The stained nuclear fraction can be resuspended in buffer and read at 355/465 nm. The cadaverine labeled pellet can be washed twice in iced buffer and resuspended in 350 μl of buffer. For each condition triplicate, 100 μl aliquots can be placed in a black 96-well plate and read on the microplate spectrophotometer. CADAVERINE 488™ labeled samples will be read at 495/519 nm, while samples stained with FITC or Texas Red compounds can be read at their appropriate wavelengths. The remaining sample can be used for Bradford protein assays and the number of nuclei and the protein concentration for each sample used as loading controls, e.g., as described by Perry (2009) supra; Yitzhaki (2009) supra.

In one embodiment, dyes to be used in exemplary compositions of this invention are screened with a fluorescence activated cell sorting (FACS) system; FACS is a technique that is used to count, characterize and sort an aqueous suspension of microscopic particles, including cells or organelles. In alternative embodiments, fluorescence-based flow cytometers are used to analyze several thousand particles per second and/or to actively separate and isolate particles that are marked or have specified properties. In alternative embodiments, FACS-based methodology and the use of commercially available cadaverine dyes can be used as a quantitative technique to measure autophagy and to collect AV for further biochemical analysis.

In alternative embodiments, known procedures for inducing autophagy, tissue homogenization and cellular lyses are used and/or adapted for the fluorescent plate reader assays, e.g., as described by Perry (2009) supra. In alternative embodiments, samples from a variety of biological sources are stained with cadaverine-based or FITC/TexasRed dye-comprising compounds of this invention. The suspension of cells or organelles processed using fluorescence activated BD FACSARIA™ cell sorter system (BD Biosciences, San Jose Calif.). Those biological samples that are positive for AV can be sorted based upon their specific light scattering and fluorescent intensity quantified and AV concentrated and selectively collected using this “high-throughput” detection system. Selected AV marked with the different compounds can then be used in Proteomic or Lipidomics analyses including e.g. electrospray ionization mass spectrometry (ESI-MS) and/or matrix-assisted laser desorption/ionization MALDI-Time-of-flight MS (MALDI-TOF-MS).

In one embodiment, a mammalian-based high-throughput assay system is used, e.g., using a stable transformed mouse that expresses both the GFP-LC3 and Cherry-LC3 proteins. In one embodiment, these or other animal models could be used to screen compounds of this invention for AV specific staining. Analogous cell line strains also can be used.

Another concern is potential cytotoxicity effect of compounds of this invention in studies that require cell viability, e.g., for their use in FACS or in vivo. For example, a cytotoxicity effect would interfere with certain applications such as FACS) or screens where stained cells are cloned or cultured for extended periods of time. Assays that screen for cytotoxicity can be used to identify any problems caused by a compound of the invention, e.g., by a dye component of a compound of the invention.

Depending on the exemplary fluorophore, the stability or emission spectra of a particular compound may not be sufficient for a particular imaging application, e.g., a plate reader, FACS or transmission electron microscopy (TEM). In one alternative embodiment, a solution is the redesign of the dye moiety with a different fluorophore, e.g., one that is brighter and/or more stable in biological context, such as e.g., Oregon Green (e.g. OREGON GREEN 488™ or OREGON GREEN 514™ (Molecular Probes, Eugene, Oreg.). Our primary concern for detailed imaging applications is to identify those dyes that label AV in fixed samples. This ability would not only allow for detailed ICH analyses of protein and vesicle profiles but could also be developed into diagnostic tools for medical applications. At this time we do not have sufficient information to predict and design which compound will meet this requirement. However, we are systematically examining the staining profile of each compound using standard fixing conditions. We will also test the staining profile of compounds using other preparation methods (stain then fix) or fixation (methanol).

In alternative embodiments, compositions of the invention comprise AV dyes that can detect autophagy or autophagosomes (AV) pathway changes under a variety of physiological conditions, e.g., including dyes to study autophagy in cardiomyoctes and ischemia/reperfusion injury models (I/R). In cardiac tissue autophagy occurs constitutively but can undergo dramatic induction following different physiological conditions like starvation or ischemia-reperfusion injury (IR). Under some physiological conditions the pathway appears to be a cardioprotective response (IR injury), protecting cardiomyocytes from hypoxia and nutrient loss. Conversely the pathway has been implicated as a negative factor during heart failure that is caused by pressure overload and tissue remodeling. Thus, in one embodiment, compositions of the invention are used to study and measure autophagy and the autophagosome (AV) pathway in the cardiac system under normal and pathological situations, e.g., during a cardioprotective response as a sequelae to IR injury.

Cardiac Cell Culture and Transfection Techniques. In one embodiment, an imaging analysis of autophagy in the cardiac system that involves the HL-1 cardiac cell line is used. Simulated ischemia/reperfusion (SI/R) HL-1 cells can be plated in gelatin/fibronectin coated 14-mm diameter glass bottom micro-well dishes (e.g., from MatTek Corp., Ashland, Mass.), and ischemia started by exchange cells into ischemia-mimetic buffer solution (125 mM NaCl, 8 mM KCl, 1.2 mM KH₂PO₄, 1.25 mM MgSO₄, 1.2 mM CaCl₂, 6.25 mM NaHCO₃, 5 mM sodium lactate, 20 mM HEPES, pH 6.6); e.g., as described by Hamacher-Brady (2002) J. Biol. Chem. 281:29776-29787; 26, 70). Dishes can be placed in hypoxic pouches (GASPAK EZ™, BD Biosciences) that are equilibrated with 95% N₂, 5% CO₂. After 2 hr of ischemia, reperfusion can be initiated by exchange cells into normoxic Krebs-Henseleit buffer solution (110 mM NaCl, 4.7 mM KCl, 1.2 mM KH₂PO₄, 1.2 mM MgSO₄, 1.2 mM CaCl₂, 25 mM NaHCO₃, 15 mM glucose, 20 mM HEPES, pH 7.4) and incubation in 95% room air, 5% CO₂. Controls can be run in parallel for each condition and time point by incubating cells in normoxic buffer. The construction of the mCherry-LC3 expression vector has been described; it can be transfected into HL-1 cells for 48 h followed by to SI/R, e.g., as described in Hamacher-Brady (2006) J. Biol. Chem. 281:29776-29787; Iwai-Kanai (2008) supra; Yitzhaki (2009) supra. Cells can be stained with the different AV dyes and fixed and non-fixed cells (4% PFA) can be examined using standard fluorescent microscopy. To quantify the autophagic response for a given condition cells can be classified as having predominantly a diffuse mCherry-LC3 fluorescence or numerous mCherry-LC3 and dye labeled puncta.

Primary cardiomyocytes studies. Adult rat cardiomyocytes can be isolated from young male Sprague Dawley rats, using standard methods, e.g., as described in Baines (2005) Nature 434: 658-662; Gottlieb (2003) Arch. Biochem. Biophys. 420:262-267; Kavazis (2008) Am. J. Physiol. Heart Circ. Physiol. 294:H928-935. Animals will be anesthetized and all animal procedures can be in accordance with the NIH guidelines and approved by the SDSU Institutional Animal Care and Use Committee. After an injection of heparin (100 U/kg) into the hepatic vein, the heart can be excised and the aorta cannulated. The heart can be perfused retrogradely with a Ca-free buffer followed by perfusion with 0.6 mg/mL collagenase (CLS 2, Worthington Biochemical Corporation, USA) and CaCl₂ in the perfusion buffer (15 min). The heart can be minced and the myocytes filtered through gauze. Protease activity can be stopped using 5% FBS and 12.5 μM CaCl₂ solution and cells were centrifuged at 1000×g for 1 min. The cell pellet can be washed in M199 medium (Invitrogen), containing 10 mM HEPES, 5 mM taurine, 5 mM creatine, 2 mM carnitine, 0.5% BSA and 100 U/mL penicillin-streptomycin. The cardiomyocytes can be plated on laminin-coated dishes (Roche) between 5-9×10⁴ cells per dish and incubated in a 5% CO₂ incubator at 37° C. Following a 24 hr recovery period cardiomyocytes can be used with various experimental conditions like amino acid deprivation, hypoxia and treatment with a range of drugs. Myocytes can be stained with the different fluorescent dyes and the number of AV and autophagic response determined using fluorescent microscopy, as described e.g., by Gottlieb (2003) supra; Baines (2005) supra.

In alternative embodiments, compositions and methods of the invention are used to detect autophagic (e.g., AV) changes associated with neurodegeneration and protein aggregates, e.g., protein aggregates in nervous or CNS tissue. There is growing body of data showing that autophagy plays a critical part in neurodegenerative disorders; protein aggregate accumulation in nerve or CNS tissue can be associated with a dramatic alteration in AV profiles. Which cytological alteration precedes the other is still hotly debated but the compositions of the invention comprising AV-selective dyes can be used to address these critical questions. The inability of neurons to mount an effective autophagic response and eliminate cytotoxic aggregates, damaged organelles and age-dependent ROS associated damage is likely a key factor in progressive neural decline and cell death; and in alternative embodiments compositions of the invention are used to assess the AV status of these neurons.

In alternative embodiments, compositions and methods of the invention are used to stain neural cells lines. In one study, the number of Texas Red+ vesicles in untreated HT22 cells was unexpected but may be consistent with other data showing basal rates of autophagy are high in neurons, see e.g., Soucek (1976) Recent Adv. Stud. Cardiac Struct. Metab. 12:453-463. In alternative aspect of these studies, neuronal cells are treated with several different compounds that activate (rapamycine) or suppress (bafilomycin and chloroquine) autophagic function. Cells also can be deprived of amino acids and exposed to hypoxia and hydrogen peroxide, e.g., as described by Simonsen (2008) supra; Soucek (2003) Neuron 39:43-56. Compositions of the invention comprising these dyes also can be used to further characterize the association of AV with protein aggregates in MC65 cells, e.g., as described by Maezawa (2006) J. Neurochem. 98:57-67; Sebastia (2006) J. Neural Transm. 113:1837-1845. In alternative embodiments, neuronal tissues samples are dyed with compositions of the invention that stain fixed or embedded tissue preparations.

In alternative embodiments, compositions and methods of the invention are used to evaluate the effect of bacterial infection and toxins on AV formation; and the correlation between infection and a cell's response. In one aspect, compositions and methods of the invention are used to evaluate the pathogenesis of bacterial meningitis, e.g., interactions between Group B Streptococcus (GBS) and brain microvascular endothelial cells (BMEC), that comprise the human blood-brain barrier. In one aspect, compositions and methods of the invention are used to characterize autophagy and AVs in the pathogenesis of an infectious disease, e.g., including bacterial, viral and parasitic infections.

In one embodiment, compositions and methods of the invention are used in co-localization analyses of fluorescent-labeled bacteria and AV; e.g., involving confocal imaging of infected samples. These studies can characterize phagocytosis, the first-line of an innate immune response, which can be triggered by infectious particles binding to specific membrane receptors (i.e. Fcγ receptors). Phagocytosis of invading pathogens can be triggered in part by engagement of the Toll-like receptor pathway signaling (TLR). Activation of TLR has been shown to recruit the LC3 protein to phagosomes thus promoting their maturation and ability to kill invading bacteria. Thus, compositions and methods of the invention are used to characterize the exact relationship between TLR signaling, phagocytosis and activation autophagy. Wild type GBS strains can be used to adhere to and invade lung epithelial cells, brain microvascular endothelial cells (BMEC) and murine macrophages. Compositions of the invention can be used with fluorescently tagged microbes and GFP expressing bacteria to examine the intracellular activities of pathogens by e.g. fluorescent microscopy.

Compositions of the invention also can be used to study the pathogenesis of bacterium, including detecting autophagic changes linked to bacterial infection, e.g. an infection by a Bacillus such as Bacillus anthracia, a Gram-positive spore-forming bacterium that causes anthrax in humans and animals. Exposure to anthrax lethal factor (LF) directly stimulates autophagy and induces the rapid formation of AV (see e.g., Tan (2009) Biochem Biophys Res Commun 379:293-297). LF has been shown to inhibit a variety of immune cells including macrophages, dendritic cells, neutrophils, T- and B-cells. In one embodiment, murine macrophage cells and human promyelocytic leukemia cells (e.g., HL-60) will be directly exposed to anthrax LF (Lists Biological, Campbell, Calif.), e.g., as described by Mock (2001) Annu. Rev. Microbiol. 55: 647-671, Tan(2009) supra; van Sorge, et al., PLoS ONE 3: e2964, 2008. Cells can be stained with compositions of this invention and imaged for altered AV and lysosomal profiles using standard sample preparation and confocal imaging techniques. Other infectious conditions and bacterial types can also be used.

Compositions of the invention also can be used to study the pathogenesis of viral infection, including detecting autophagic changes linked to viral infection, including acute and persistent RNA virus infections and host-viral pathogen interactions which activate both the innate and adaptive immune responses. Compositions of the invention can be used with cultured and in vivo models of infection, e.g., using a pathogenic human strain of coxsackievirus B3 (CVB3; which are ubiquitous pathogens that are associated with several human diseases, including pancreatitis, myocarditis, diabetes, and aseptic meningitis. Compositions of the invention also can be used to study the pathogenesis of lentiviruses, e.g., HIV. HIV is associated with dementia (called HAD in monkeys) and has been linked to the inhibition of neuronal autophagy, suggesting the pathway is a protective mechanism for latently infected neurons. As with bacterial factors exposure of non-infected cells to HIV-1 envelope glycoproteins results in the up regulation of autophagy and the eventual triggering of cellular apoptosis.

Compositions of the invention can be used to investigate the complex relationship that viral protein exposure and infections can have on the regulation of autophagy and AVs. For example, compositions of the invention can be used to investigate the autophagic response of differentiated and non-differentiated neurospheres to viral (e.g., CVB3) infection. For example, cell types can be transduced with a GFP-LC3 construct and infected with dsRED-labeled-CVB3 and cultured with compositions of the invention and be observed by fluorescence microscopy, e.g., as described by Feuer (2003) Am. J. Pathol. 163:1379-1393. In one study (cell types transduced with GFP-LC3 construct and infected with dsRED-labeled-CVB3) the percentage of GFP-positive cells with abundant GFP-puncta was determined for each condition and the undifferentiated neurospheres were found to have a significant increase in AV numbers (Feuer (2003) supra). Infected and mock-treated cells can be stained with compositions of this invention and counterstained e.g. with LYSOTRACKER™ and/or HOECHST 33342™ (Invitrogen). Compositions of this invention also can be used to determine AV infection profiles generated by other viral types and in additional cell lines.

Compositions of this invention may have a non-specific staining pattern or have unpredicted interactions with pathogens, e.g., in viral and bacterial infections. Depending on which cultured cell type or animal model system, dye-comprising compositions of this invention may also cause a non-specific alteration of autophagy and alter in AV profiles without infection. Both concerns require us of appropriate control assays that include e.g. an examination of individual dye staining patterns with that of pathogen and host strains that will be used for a given experiment. Before pathogen assays begin any effects the dye-comprising compositions of this invention cause in long-term changes to AV profiles can be established. In general cells or whole tissues can be exposed to dye-comprising compositions of this invention for between about 1 to 3 days. The dye-comprising compositions of this invention can be re-applied along with fresh staining with commercially available dyes, e.g., LYSOTRACKER™ of BODIPY-cadaverine.

In alternative embodiments, compositions of this invention are used to characterize the sub-cellular distribution of AV and/or non-AV labeling dyes. This takes advantage of compounds of this invention that highlight subcellular structures or vesicles within cells, but are not specific for the autophagic-lysosomal populations of organelles. Compositions of this invention also can be used to locate (e.g., map) organelles such as early-late endosomes, peroxisomes, mitochondria, endoplasmic reticulum and Golgi apparatus, and to characterize their staining patterns. In one embodiment, direct staining with compositions of this invention and immunocytochemistry are used fresh and fixed cells to mark different vesicle types. Samples can be examined by confocal microscopy and high-resolution images generated to show co-localization of the different fluorophores. Organelle-specific markers that can be used with compositions of this invention include for example:

TABLE 1
Additional SelectiveMarkers for Subcellular Organelles

| Fluorescent Markers | Organelle |
|---|---|
| pGFP-EEA1 delta1-1256Q | EE |
| pEGFP EEA1 | EE |
| pEGFP-2xFyve | PI3P; EE |
| pEGFP-Rab7 | LE |
| PEGFP-Rab4a | RE |
| PEGFP-Rab5a | EN Pathway |
| pEGFP-CD 63 | LE |
| pEGFP-EGFR | EN Pathway |
| pDest-Cherry-GFP-2xFyve | PI3P: EEZ: AV |
| pDEST-Tomato-2x FYVE | PI3P: EEZ: AV |
| pDEST-Tomato-EEA1-CT | EE |
| pEGFP-C1-hApg5 | AV |
| pEGFP-C1-hLC3 | AV |
| pEGFP-p62 | AG: AV |
| pDEST-Cherry-GFP-LC3B | AV |
| pDEST-Cherry-GFP-p62 | AG: AV |
| pDEST-Tomato-p62 | AG: AV |
| GFP-hAtg5 K130R-HA | Dominant Neg. |
| Dyes/Stains | Organelle |
| Lysotracker Red | Lys; AV |
| Lysotracker Green | Lys; AV |
| MitoTracker Red | Mito |
| MitoTracker Green | Mito |
| Phalloidin Green | Actin |
| Drosophila Markers | Organelle |
| UAS-pGFP-Atg8a | AV |
| UAS-pGFP-Atg5 | AV |
| UAS-Cherry-Atg8a | AV |
| UAS-pGFP-Ref(2)P | AV |
| UAS-pGFP-Rab11 | EN Pathway |
| UAS-pYFP-Golgi | Trans-Golgi |
| UAS-pYFP-ER | Endo. Retic. |
| UAS-pGFP-Golgi | Golgi Net. |
| UAS-pYFP-synapse | synaptic vesicle |
| UAS-GFP-CT-LAMP | Lys |
| UAS-Caxx-GFP cyto | Cyto. Mem. |
| mem | |
| Primary Antibodies | Host Species |
| hAlfy | Rabbit |
| dBchs | Rabbit |
| Mammalian p62 | Guinea Pig |
| dRef(2)P/p62 | Rabbit; Rat |
| dRab11 | Rabbit |
| dAtg8a (hGABARAP) | Rabbit |
| hLC3 | Rabbit; Mouse |
| Ubiquitin (mam & fly) | Rabbit; Mouse |
| hAtg5 | Rabbit |
| Actin (mam & fly) | Mouse |
| hLAMP-I | Mouse |
| hLAMP-II | Mouse |

EE = early endosomes
RE = recycling endosomes
Lys = Lysosomes
Mito = mitochondria
LE = late endosomes
AV = autophagic vesicles

A number of embodiments of the invention have been described. Nevertheless, it will be understood that various modifications may be made without departing from the spirit and scope of the invention. Accordingly, other embodiments are within the scope of the following claims.

1. A chimeric molecule comprising:
(1)(i) at least two domains (or moieties, or groups) comprising:
(a) a first domain or moiety (or group) comprising: a primary amine; a bifurcated di- or triamine; a tertiary amine; a polyamine; an N,N-dimethyl or diethyl amine; an aliphatic amine; a heteroaromatic amine; an ethylenediamine; a 1,3-diaminopropane; a 1,4-diaminobutane; a 1,6 diaminohexane; a 2,2′ (ethylenedioxy)diethylamine; a triethylene glycol diamine; an N,N-dimethylaniline; a guanidine; a spermine or a spermidine (linear); or a structure selected from the group consisting of 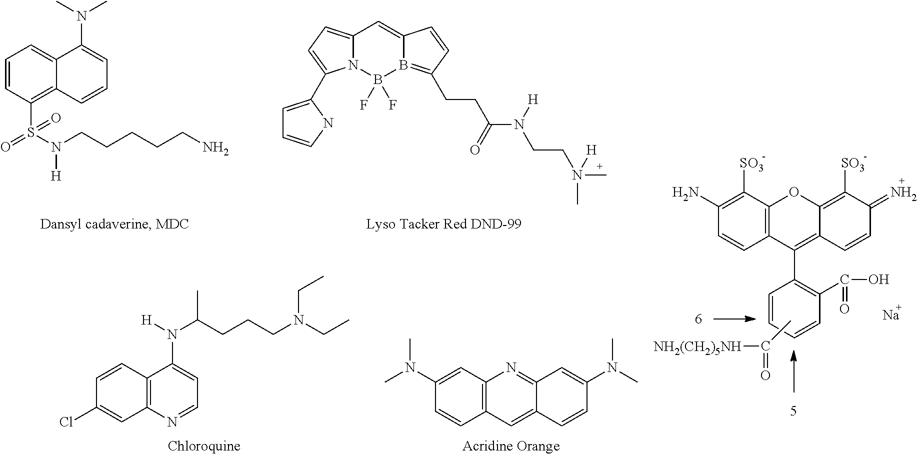
AlexaFluor 488™ cadaverine, or other fluor-conjugated cadaverine molecules (see list)
Alexa Fluor® 647 azide, triethylammonium salt
Alexa Fluor® 350 cadaverine
Alexa Fluor® 405 cadaverine, disodium salt
Alexa Fluor® 488 cadaverine, sodium salt
Alexa Fluor® 555 cadaverine, disodium salt
Alexa Fluor® 568 cadaverine, diammonium salt
Alexa Fluor® 594 cadaverine
Alexa Fluor® 647 cadaverine, disodium salt
fluo-4 cadaverine, pentapotassium salt
Oregon Green® 488 cadaverine *5-isomer*
Texas Red® cadaverine (Texas Red® C₅)
5-(((4-(4,4-difluoro-5-(2-thienyl)-4-bora-3a,4a-diaza-s-indacene-3-yl)phenoxy)acetyl)amino)pentylamine, hydrochloride (BODIPY® TR cadaverine),
or equivalents thereof, or derivatives thereof, or any combination thereof; and
(b) a second domain or moiety (or group) comprising a detectable or “reporter” composition or moiety; and
(ii) a spacer, linker or direct coupling agent covalently or non-covalently joining the first domain or moiety to the second domain or moiety,
wherein the chimeric molecule is capable of localizing to (detecting, or binding to) an autophagosome (or autophagic vesicle, or AV) to detect and/or measure the amount of autophagic activity in a cell extract, a cell, a tissue, an organ or an organism,
wherein optionally the chimeric molecule is capable of localizing to (detecting, or binding to) an AV sub-populations detect and/or measure the amount of the AV subpopulation,
wherein optionally the AV subpopulation comprises an autophagosome AV subpopulation, an autolysosome AV subpopulation or a lysosomal vesicle AV subpopulation;
(2) the chimeric molecule of (1), wherein the detectable or “reporter” composition or moiety comprises a radioactive, a radio-opaque, a fluorescent, bioluminescent and/or paramagnetic composition or moiety, or heavy metals for TEM, or equivalents thereof, or derivatives thereof, or any combination thereof;
(3) the chimeric molecule of (1) or (2), wherein the detectable or “reporter” composition or moiety comprises a dansyl, a monodansyl, a fluorescein, a fluorescein isothiocyanate (FITC), a boron-dipyrromethene (BODIPY, or 4,4-difluoro-4-bora-3a,4a-diaza-s-indacene), a BODIPY-TR™, an ALEXA FLUOR™ dye (Molecular Probes, Life Sciences, Carlsbad, Calif.), an ALEXAFLUOR488™, a DYLIGHT™ fluor (Thermo Fisher Scientific, Waltham, Mass.), a DYLIGHT 488™ fluor, an ATTO™ dye (ATTO-TEC, GmbH, Siegen, Germany), a HILYTE dye (AnaSpec Inc., San Jose, Calif.), a positron-emitting agent, a Fluorine-18, a Carbon-11, a quantum dot nanoparticle, a gadolinium, a ferritin or nanoparticles of heavy metals; or equivalents thereof, or derivatives thereof, or any combination thereof;
(4) the chimeric molecule of (1), (2) or (3), wherein the spacer, linker or direct coupling agent comprises a peptide or a synthetic molecule, or the spacer, linker or direct coupling agent comprises a thiourea, a sulfonamide or an amide; or equivalents thereof, or derivatives thereof, or any combination thereof,
(5) the chimeric molecule of any of (1) to (4), wherein the peptide or synthetic molecule comprises a polyglycine; a polyethylene glycol; a peptide comprising glycine, serine, threonine and/or alanine; a carbodiimide; a sulfhydryl-reactive composition; a glutaraldehyde or a glutardialdehyde (pentanedial); a hetero-bifunctional photoreactive phenylazide; a N-hydroxy-succinimidyl-comprising composition; or equivalents thereof, or derivatives thereof, or any combination thereof; or a structure selected from the group consisting of: 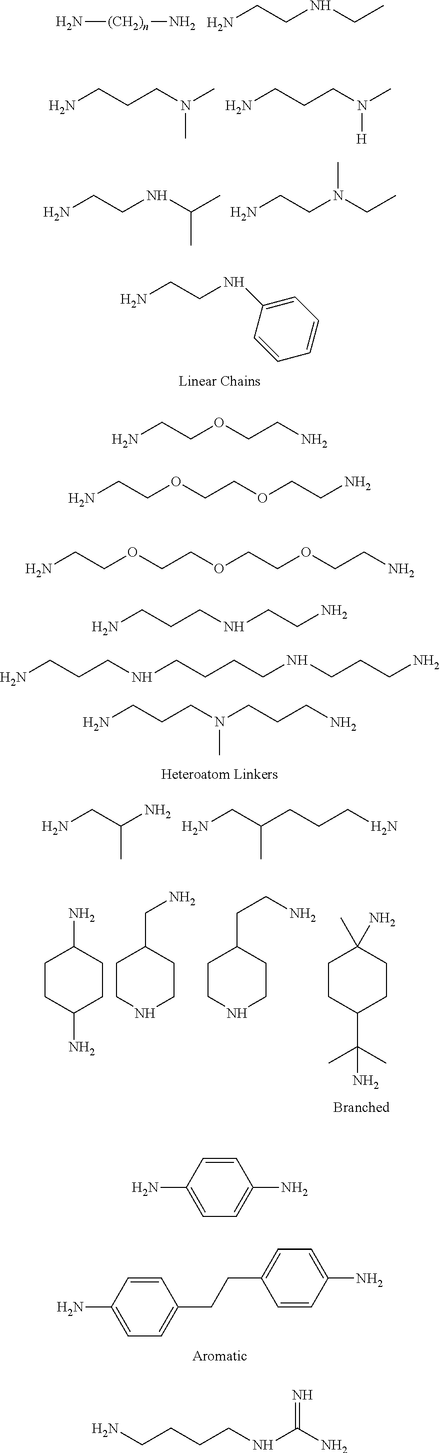 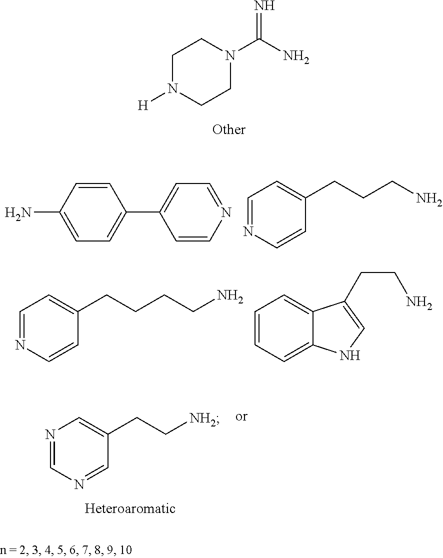
or
(6) the chimeric molecule of any of (1) to (5), wherein: the carbodiimide comprises dicyclohexylcarbodiimide (DCC), diisopropylcarbodiimide (DIC) or N′-(3-dimethylaminopropyl)-N-ethylcarbodiimide hydrochloride (EDC); or the sulfhydryl-reactive composition comprises a maleimide, a pydridyldisulfide, an alpha-haloacetyl, a vinylsulfone or a sulfatoalkylsulfone; the hetero-bifunctional photoreactive phenylazide comprises a sulfosuccinimidyl-2-(p-azido salicylamido)ethyl-1,3′-dithiopropionate; the N-hydroxy-succinimidyl-comprising composition comprises N-Succinimidyl-5-acetylthioacetate (SATA), an N-Succinimidyl 3-(2-pyridyldithio)-propionate) (SPDP), a Succinimidyl 6-(3-[2-pyridyldithio]-propionamido)hexanoate) (LC-SPDP), or an (N-Succinimidyl[4-iodoacetyl]aminobenzoate) (SIAB); or equivalents thereof, or derivatives thereof, or any combination thereof.

2-6. (canceled)

7. A liposome, pharmaceutical composition or formulation, inhalant or spray formulation, or parenteral or enteral formulation, comprising:
(a) the chimeric molecule of claim 1 formulated with a pharmaceutically acceptable excipient; or
(b) the liposome, pharmaceutical composition or formulation, inhalant or spray formulation, or parenteral or enteral formulation of (a), wherein the enteral formulation is formulated for oral, rectal or sublingual administration or for intravenous, subcutaneous, intrathecal or intramuscular administration.

8-11. (canceled)

12. A method for detecting or measuring the amount of autophagic activity in a cell extract, a cell, a tissue, an organ or an organism, or detecting or binding or measuring the amount of to an autophagosome (or autophagic vesicle, or AV), in a cell extract, a cell, a tissue, an organ or an organism, comprising:
(i)(a) providing a cell extract, a cell, a tissue, an organ or an organism and the chimeric molecule of claim 1;
(b) contacting the chimeric molecule with the cell extract, cell, tissue, organ or organism; and
(c) detecting the presence and amount of the detectable composition or moiety; and optionally further comprising detecting the location of the chimeric molecules in the cell extract, cell, tissue, organ or organism,
wherein optionally the chimeric molecule is capable of localizing to (detecting, or binding to) an AV sub-population to detect and/or measure the amount of the AV subpopulation,
wherein optionally the AV subpopulation comprises an autophagosome AV subpopulation, an autolysosome AV subpopulation or a lysosomal vesicle AV subpopulation;
(ii) the method of (i), wherein the detecting step (c) comprises (a) use of a fiberoptic catheter or needle comprising a detecting device for detecting and measuring the amount of the detectable composition or moiety in a cell, tissue, organ or organism, and/or comprises use of a fluorimeter or luminometer attached to a fiberoptic probe;
(iii) the method of (i) or (ii), wherein the method comprises (a) use of a paramagnetic agent injected into a cell, tissue, organ or organism, and the amount of the detectable composition or moiety incorporated into the cell, tissue, organ or organism is an indicator of the extent of autophagy in that site; (b) the method of (a), wherein the amount of the detectable composition or moiety is assessed (measured) using nuclear magnetic resonance (NMR or MRI) imaging; or (c) the method of (a) or (b), wherein the detectable composition or moiety comprises a gadolinium or a ferritin;
(iv) the method of (i), (ii) or (iii), wherein the method comprises or further comprises: (a) the detectable composition or moiety comprises a positron-emitting agent injected into a cell, tissue, organ or organism, and the amount of the detectable composition or moiety incorporated into the cell, tissue, organ or organism is an indicator of the extent of autophagy in that site; (b) the method of (a), wherein the amount of the detectable composition or moiety is assessed (measured) using a positron emission tomography (PET) imaging; or (c) the method of (a) or (b), wherein the detectable composition or moiety comprises a Fluorine-18 or a Carbon-11 incorporated into the moiety; or
(v) the method of any of (i) to (iv), wherein the cell, tissue, organ or organism sample is or comprises a biopsy sample and/or a cell extract.

13-16. (canceled)

17. A method for the high-throughput screening of drugs or reagents that modulate autophagy or the amount of autophagosomes (AV) in a cell extract, cell, tissue, organ, organism or individual, comprising:
(i)(a) providing the chimeric molecule of claim 1;
(b) providing a test reagent or drug (a candidate drug or reagent to be screened for its ability to modulate autophagy);
(c) contacting one sample of (or derived from) a cell extract, cell, tissue, organ, organism or individual with the chimeric molecule (control sample), and contacting a second sample (equivalent to the first sample for comparative purposes) with the test reagent or drug and the chimeric molecule (test sample); and
(d) detecting the amount of autophagy, or the amount of autophagosomes, in the cell extract, cell, tissue, organ, organism or individual with and without the test reagent or drug,
wherein an increase or a decrease in the amount of autophagy as compared to control (without test reagent or drug) indicates that the test reagent or drug is a modulator of autophagy in a cell extract, cell, tissue, organ, organism or individual,
wherein an increase or a decrease in the amount of the detectable composition or moiety as compared to control (without the detectable composition or moiety) in a cell extract, cell, tissue, organ, organism or individual indicates that the test reagent or drug is a modulator of autophagy in the cell extract, cell, tissue, organ, organism or individual;
(ii) the method of (i), wherein fluorescence microscopy or a fluorescence imaging system is used to determine the amount of and/or the location of the detectable composition or moiety in the cell extract, cell, tissue, organ, organism or individual; or
(iii) the method of (i) or (ii), wherein the screening comprises high-content imaging on a multi-well plate; or
(iv) the method of any of (i) to (iii), wherein the screening is constructed and practiced on a multi-well plate; or
(v) the method of any of (i) to (iv), wherein transmission electron microscopy (TEM) is used to determine the amount of and/or the location of the detectable composition or moiety in the cell extract, cell, tissue, organ, organism or individual.

18-21. (canceled)

22. A method for assessing (evaluating) the efficacy of a therapeutic or prophylactic (test) drug or composition by assessing its ability to modulate autophagy or modulate the amount of autophagosomes (AV) in a cell extract, cell, tissue or organism or individual, comprising:
(i)(a) providing the chimeric molecule of claim 1;
(b) providing a therapeutic or a prophylactic drug or composition;
(c) contacting one sample of a cell extract, cell, tissue, organ or organism or individual with the chimeric molecule (control sample), and contacting a second sample (equivalent to the first sample for comparative purposes) with the therapeutic or prophylactic drug (test) drug and the chimeric molecule (test sample); and
(d) detecting the amount of autophagy in the cell extract, cell, tissue, organ or organism or individual with and without the test reagent or drug,
wherein an increase or a decrease in the amount of autophagy as compared to control (without test reagent or drug) indicates that the test reagent or drug is a modulator of autophagy in a cell extract, cell, tissue, organ or organism or individual,
wherein an increase or a decrease in the amount of detectable composition or moiety as compared to control (without detectable composition or moiety) in a cell extract, cell, tissue, organ or individual indicates that the test reagent or drug is a modulator of autophagy in the cell extract, cell extract, cell, tissue or organ or individual;
(ii) the method of (i), wherein the method assesses (evaluates) the efficacy of a therapeutic or prophylactic (test) drug for treating, ameliorating or preventing myocardial ischemia/reperfusion injury, a neurodegenerative disease, diabetes, atherosclerosis, cardiac hypertrophy, heart failure, glycogen storage disease type II (also called Pompe disease or acid maltase deficiency) and related conditions;
(iii) the method of (ii), wherein the neurodegenerative disease is Alzheimer's disease, Lewy Body Disease, Parkinson's Disease, Huntington's Disease, Multi-infarct dementia, senile dementia or Frontotemporal Demential;
(iv) the method of (ii), wherein the neurodegenerative disease is related to or is a sequelae of a trauma, or exposure to a toxin or a poison;
(iv) the method of (ii), wherein fluorescence microscopy or a fluorescence imaging is used to determine the amount of and/or the location of the detectable composition or moiety in the cell extract, cell, tissue or organ; or
(v) the method of (iv), wherein transmission electron microscopy (TEM) is used to determine the amount of and/or the location of the detectable composition or moiety in the cell extract, cell, tissue or organ.

23-27. (canceled)

28. A kit comprising: (a) the composition of claim 1; or (b) the kit of (a), further comprising instruction for practicing a method for detecting or measuring the amount of autophagic activity in a cell extract, a cell, a tissue, an organ or an organism, or detecting or binding or measuring the amount of to an autophagosome (or autophagic vesicle, or AV), in a cell extract, a cell, a tissue, an organ or an organism.
